(19) United States
(12) Patent Application Publication
(10) Pub. No.: US 2013/0104445 A1
(43) Pub. Date: May 2, 2013

(54) ESSENTIAL OILS AND THEIR COMPOUNDS AS STINK BUG REPELLENTS

(71) Applicants: Rodney G. Schneidmiller, Greenacres, WA (US); Qing-He Zhang, Spokane Valley, WA (US)

(72) Inventors: Rodney G. Schneidmiller, Greenacres, WA (US); Qing-He Zhang, Spokane Valley, WA (US)

(21) Appl. No.: 13/661,217

(22) Filed: Oct. 26, 2012

Related U.S. Application Data
(60) Provisional application No. 61/552,935, filed on Oct. 28, 2011.

(51) Int. Cl. A01M 1/02; A01M 1/10; A61L 9/04

(52) U.S. Cl. 43/107; 239/6

(57) ABSTRACT

Provided herein are essential oils and synthetic compounds, and combinations thereof, as repellent compositions for repelling insects, such as members of the Pentatomoidea superfamily, the Superfamily Coreoidea, and the family Miridae, commonly referred to as stink bugs/shield bugs, squash bugs and plant bugs, respectively. Controlled release devices comprising these repellent compositions are also provided. The repellent compositions may additionally be combined with attractant compositions in a type of “push-pull” method. The use of both repellents and attractants can increase the success rate for controlling stink bugs and the like.

### CROSS-REFERENCE TO RELATED APPLICATION

This application claims the benefit of U.S. Provisional Application No. 61/552,935, filed Oct. 28, 2011, incorporated herein expressly by reference.

### BACKGROUND

Pesticides, such as insecticides, are commonly used in agricultural, industrial, and residential settings to battle destructive, pestiferous, and/or disease-carrying insects and other arthropods. Insecticides have achieved significant successes in controlling pestiferous and disease-vector arthropods and have increased in their lethality over the years. However, increasingly the environmental and human health effects of pesticides, as well as their deleterious effects on beneficial insect species and other animals, have caused users to seek other means for controlling pest insect populations.

For example, many stink bugs, shield bugs, squash bugs, and plant bugs are considered agricultural pest insects. They can generate large populations that damage crop production, and are resistant to many pesticides. Moreover, they are immune to the GM (genetically modified) crops, such as the transgenic Bt (Bacillus thuringiensis) crops. Over the past 5-10 years, the stink bugs and plant bugs have become the most serious pest problems in many parts of the world, especially in the regions with large areas of Bt crops.

For example, the brown marmorated stink bug (BMSB), the Halyomorpha halys (Stal) native to Asia, is believed to have been accidentally introduced into the United States as early as 1996, likely as stowaways, possibly as eggs, on packing crates, or the like. The brown marmorated stink bug has been recorded in a total of 33 states and the District of Columbia, according to information provided by the National Agricultural Pest Information System (NAPIS) (http://pests.ceris.purdue.edu). In 2010, the BMSB emerged as a severe pest of fruit and other crops across the region. In addition, this invasive species is a serious nuisance for homeowners and businesses as it overwinters in residential houses, commercial buildings, and warehouses.

The brown marmorated stink bug can cause widespread damage to fruit, vegetable, and field crops, including peaches, apples, cherries, raspberries, pears, tomatoes, green beans, soybeans, and corns, among many others. It is a sucking insect that uses its proboscis to pierce the host plant in order to feed. This feeding may cause necrotic areas on the outer surface of fruits, leaf stippling, cat-facing on tree fruits, seed loss, and transmission of plant pathogens. Frequently, the brown marmorated stink bug survives the winter as an adult by entering structures that shield them from the elements. Inside a house they may go into a state of hibernation where they wait for winter to pass.

Research efforts to develop control or management strategies for these pestiferous stink bugs have focused on pheromone or kairomone attractants for traps to monitor or reduce the stink bug populations (both adults and nymphs). Several commercial traps baited with stink bug pheromones have been promoted and used in recent years as effective tools for monitoring or controlling the stink bugs, including the Rescue® stink bug traps (patent Nos. U.S. D645,534S and U.S. D645,535S). In addition, several sticky or non-sticky traps equipped with various wave-lengths of lights have been developed and commercialized recently for capturing the overwintering adults indoors, such as the Rescue® stink bug lights.

In contrast to the intensive work and significant progress on the pheromone/kairomone-baited traps or light traps, which target stink bug population reduction (an indirect protection measure), little or no research effort has been made on developing direct protection tactics, namely, the stink bug repellents. A stink bug repellent was recently reported by Nakajima et al. in 2010 [Nakajima et al. 2010. 3-(4-Methylfuran-3-yl)propan-1-ol: a white-spotted stinkbug (Eysarcoris ventralis) repellent produced by an endophyte isolated from green foxtail. J. Agric. Food Chem., 58 (5), pp. 2882-2885], based on a lab petri dish bioassay test. Unfortunately, it is still not known if this reported repellent compound and its derivatives will show significant repellency in the field yet. Therefore, to the best of the inventors' knowledge, there are no other conclusively confirmed spatial repellents for any stink bugs so far.

### SUMMARY

This summary is provided to introduce a selection of concepts in a simplified form that are further described below in the Detailed Description.

A method for repelling an insect is disclosed. The method includes releasing into a space a repellent composition comprising at least one of (a) or (b), or both (a) and (b), wherein (a) is a first essential oil selected from the group consisting of lemongrass oil, ylang ylang oil, clove oil, geranium oil, rosemary oil, spearmint oil, wintergreen oil, and pennyroyal oil, or any combination thereof, and (b) is a second essential oil comprising at least one compound selected from the group consisting of 1-menthone, P-menthone, eugenol, E-citral, Z-citral, pulegone, methyl benzoate, l-carvone, methyl salicylate, benzyl acetate, β-caryophyllene, geraniol, camphor, cis-rose-oxide, an isomer thereof, or any combination thereof, wherein the compound is a major constituent of the second essential oil. The repellent composition is comprised in a controlled release device having at least one aperture configured to achieve a desired rate of release of the repellent composition in a volatilized state into the space, and the method includes repelling from the space an insect belonging to the insect order Heteroptera, the Pentatomoidea superfamily, including stink bugs, and shield bugs; the Superfamily Coreoidea, including squash bugs, or the insect family Miridae, including plant bugs, wherein each essential oil of (a) or (b) acts to repel the insect.

The repellent composition may include spearmint oil in an amount effective to repel the insect. The repellent composition may include a combination of spearmint oil, clove oil, and lemongrass oil in an amount effective to repel the insect. The repellent composition may include l-carvone in an amount effective to repel the insect.

In the disclosed method, the repellent composition can include two or more first essential oils of (a).

In the disclosed method, the repellent composition can include two or more second essential oils of (b).

In the disclosed method, the repellent composition can include at least one first essential oil of (a) and at least one second essential oil of (b).

In the disclosed method, the repellent composition can include at least one synthetic compound selected from the group consisting of I-menthone, P-menthone, eugenol, E-citral, Z-citral, pulegone, methyl benzoate, l-carvone, methyl salicylate, benzyl acetate, β-caryophyllene, geraniol, camphor, cis-rose-oxide, an isomer thereof, or any combination thereof.

In the disclosed method, the controlled release device can include a polymeric sheet having a means for permitting the repellent composition in a volatilized state to pass therethrough.

In the disclosed method, the polymeric sheet can include a plurality of lamina.

In the disclosed method, an innermost lamina of the plurality of lamina can be semi-permeable such that the repellent composition in a volatilized state can pass through the innermost lamina.

In the disclosed method, a means for permitting the repellent composition to pass therethrough may include a plurality of micro-perforations.

In the disclosed method, a polymeric sheet can further include an innermost lamina, wherein at least some of the plurality of micro-perforations do not penetrate an innermost lamina of the polymeric sheet.

The disclosed method may further include placing the controlled release device outdoors.

In the disclosed method, the controlled release device may be placed on or near a target selected from the group consisting of vegetable garden, orchard, the eaves of a window or wall of a building structure, to prevent an insect from sensing or approaching the target.

In the disclosed method, the insect may be selected from the group consisting of stink bugs, shield bugs, squash bugs, and plant bugs or any combination thereof.

In the disclosed method, the insect may be selected from a green stink bug (Acrosternum hilare (Say)); any of several species of Euschistus spp. including a brown stink bug (Euschistus servus (Say)), E. tristigmus, E. conspersus, E. variolarius, E. politus, or E. heros; a southern green stink bug (Nezara viridula (L.)); an eastern green stink bug (Nezara antennata); a spined soldier bug (Podisus maculiventris); an East Asian stink bug, a yellow-brown stink bug, a brown marmorated stink bug (Halyomorpha halys); a red-shouldered stink bug (Thyanta pallidovirens); a globular stink bug (Megacopta punctatissimum); a white-spotted stink bug (Eysarcoris ventralis); a fruit-piercing stink bug (Glaucias subpunctatus); a red-striped stink bug (Graphosoma rubrolineatum); a brown marmorated stink bug (Halyomorpha mista); a rice stink bug (Lagynotomus elongates, Oebalus pugnax); a two-spotted stink bug (Perillus bioculatus); a conchuela stink bug (Chlorochroa ligata); a Uhler's stink bug (Chlorochroa uhlerii); a Say's stink bug (Chlorochroa sayi); a brown-winged green stink bug (Plautia stali (Scott)); a boxelder bug. (Boisea trivittata (Say)); a species of Banasa dimidiata (Say); a species of B. calva (Say); a species of B. euchlora Stål; a Harlequin bug (Murgantia histrionica); a Kudzu bug [Megacopta cribraria (Fabricius)]; a species of Anasa spp., including A. tristis (DeGeer); a species from the genus Leptoglossus; a species of Lygus spp., including a tarnished plant bug (L. lineolaris), a western tarnished plant bug (L. hesperus), a European tarnished plant bug (L. ruguilipnennis), a green leaf bug (L. lucorum); a species of apple dimpling bug (Campylomma spp.); a species of mosquito bug (Helopeltis spp.); a species of a honeylocust plant bug (Diaphnocoris spp.); a species of green mirid (Creontiades spp.); and a species of potato mirid (Calocoris spp).

A method for repelling insects is disclosed. The method includes releasing into a space a repellent composition comprising at least one synthetic compound selected from the group consisting of I-menthone, P-menthone, eugenol, E-citral, Z-citral, pulegone, methyl benzoate, l-carvone, methyl salicylate, benzyl acetate, β-caryophyllene, geraniol, camphor, cis-rose-oxide, an isomer thereof, or any combination thereof, wherein the repellent composition is comprised in a controlled release device having at least one aperture configured to achieve a desired rate of release of the repellent composition in a volatilized state into the space; and repelling from the space an insect belonging to the insect order Heteroptera, the Pentatomoidea superfamily, including stink bugs, and shield bugs; the Superfamily Coreoidea, including squash bugs, or the insect family Miridae, including plant bugs, wherein each synthetic compound acts to repel the insect.

The repellent composition may include spearmint oil in an amount effective to repel the insect. The repellent composition may include a combination of spearmint oil, clove oil, and lemongrass oil in an amount effective to repel the insect. The repellent composition may include l-carvone in an amount effective to repel the insect.

In the disclosed method, the repellent composition can further include at least one of (a) or (b),

(a) a first essential oil selected from the group consisting of lemongrass oil, ylang ylang oil, clove oil, geranium oil, rosemary oil, spearmint oil, wintergreen oil, and pennyroyal oil or any combination thereof;

(b) a second essential oil comprising at least one compound selected from the group consisting of I-menthone, P-menthone, eugenol, E-citral, Z-citral, pulegone, methyl benzoate, l-carvone, methyl salicylate, benzyl acetate, β-caryophyllene, geraniol, camphor, cis-rose-oxide, an isomer thereof, or any combination thereof, wherein the compound is a major constituent of the second essential oil.

In the disclosed method, the repellent composition may include two or more first essential oils of (a).

In the disclosed method, the repellent composition may include two or more second essential oils of (b).

In the disclosed method, the repellent composition may include at least one first essential oil of (a) and at least one second essential oil of (b).

In the disclosed method, the controlled release device may include a polymeric sheet having a means for permitting the repellent composition in a volatilized state to pass therethrough.

In the disclosed method, the polymeric sheet may include a plurality of lamina.

In the disclosed method, an innermost lamina of the plurality of lamina may be semi-permeable such that the repellent composition in a volatilized state can pass through the innermost lamina.

In the disclosed method, the means for permitting the repellent composition to pass therethrough may include a plurality of micro-perforations.

In the disclosed method, the polymeric sheet may further include an innermost lamina and wherein at least some of the plurality of micro-perforations do not penetrate an innermost lamina of the polymeric sheet.

The disclosed method may further include placing the controlled release device outdoors and/or indoors.

In the disclosed method, the controlled release device may be placed on or near a target selected from the group consisting of vegetable garden, orchard, the eaves or window or wall of a building structure, to prevent an insect from sensing or approaching the target.

In the disclosed method, the insect may be selected from the group consisting of stink bugs, shield bugs, squash bugs and plant bugs or any combination thereof.

In the disclosed method, the insect may be selected from a green stink bug (Acrosternum hilare (Say)); any of several species of Euschistus spp. including a brown stink bug (Euschistus servus (Say)), E. tristigmus, E. conspersus, E. variolarius, E. politus, or E. heros; a southern green stink bug (Nezara viridula (L.)); an eastern green stink bug (Nezara antennata); a spined soldier bug (Podisus maculiventris); an East Asian stink bug, a yellow-brown stink bug, a brown marmorated stink bug (Halyomorpha halys); a red-shouldered stink bug (Thyanta pallidovirens); a globular stink bug (Megacopta punctatissimum); a white-spotted stink bug (Eysarcoris ventralis); a fruit-piercing stink bug (Glaucias subpunctatus); a red-striped stink bug (Graphosoma rubrolineatum); a brown marmorated stink bug (Halyomorpha mista); a rice stink bug (Lagynotomus elongates, Oebalus pugnax); a two-spotted stink bug (Perillus bioculatus); a conchuela stink bug (Chlorochroa ligata); a Uhler's stink bug (Chlorochroa uhlerii); a Say's stink bug (Chlorochroa sayi); a brown-winged green stink bug (Plautia stali (Scott)); a boxelder bug (Boisea trivittata (Say)); a species of Banasa dimidiata (Say); a species of B. calva (Say); a species of B. euchlora Stål; a Harlequin bug (Murgantia histrionica); a Kudzu bug [Megacopta cribraria (Fabricius)]; a species of Anasa spp., including A. tristis (DeGeer); a species from the genus Leptoglossus; a species of Lygus spp., including a tarnished plant bug (L. lineolaris), a western tarnished plant bug (L. hesperus), a European tarnished plant bug (L. ruguilipnennis), a green leaf bug (L. lucorum); a species of apple dimpling bug (Campylomma spp.); a species of mosquito bug (Helopeltis spp.); a species of a honeylocust plant bug (Diaphnocoris spp.); a species of green mirid (Creontiades spp.); and a species of potato mirid (Calocoris spp).

A method for repelling insects is disclosed. The method includes releasing into a space a repellent composition comprising the Spined soldier bug aggregation pheromone; wherein the repellent composition comprises trans-2-hexenal, benzyl alcohol and α-terpineol in a controlled release device having at least one aperture configured to achieve a desired rate of release of the repellent composition in a volatilized state into the space, and repelling from the space an insect belonging to the insect Pentatomoidea superfamily, including stink bugs, and shield bugs; the Superfamily Coreoidea, including squash bugs, or the family Miridae, including plant bugs, wherein each synthetic compound acts to repel the insect.

In the disclosed method, the controlled release device may include a polymeric sheet having a means for permitting the repellent composition in a volatilized state to pass therethrough.

In the disclosed method, the polymeric sheet may include a plurality of lamina.

In the disclosed method, an innermost lamina of the plurality of lamina may be semi-permeable such that the repellent composition in a volatilized state can pass through the innermost lamina.

In the disclosed method, the means for permitting the repellent composition to pass therethrough may include a plurality of micro-perforations.

In the disclosed method, a polymeric sheet may further include an innermost lamina, wherein at least some of the plurality of micro-perforations do not penetrate an innermost lamina of the polymeric sheet.

The disclosed method may further include placing the controlled release device outdoors and/or indoors.

In the disclosed method, the controlled release device may be placed on or near a target selected from the group consisting of vegetable garden, orchard, the eaves or window or wall of a building structure, to prevent an insect from sensing or approaching the target.

In the disclosed method, the insect is selected from the group consisting of stink bugs, shield bugs, squash bugs, and plant bugs, or any combination thereof.

In the disclosed method, the insect may be selected from a green stink bug (Acrosternum hilare (Say)); any of several species of Euschistus spp. including a brown stink bug (Euschistus servus (Say)), E. tristigmus, E. conspersus, E. variolarius, E. politus, or E. heros; a southern green stink bug (Nezara viridula (L.)); an eastern green stink bug (Nezara antennata); a spined soldier bug (Podisus maculiventris); an East Asian stink bug, a yellow-brown stink bug, a brown marmorated stink bug (Halyomorpha halys); a red-shouldered stink bug (Thyanta pallidovirens); a globular stink bug (Megacopta punctatissimum); a white-spotted stink bug (Eysarcoris ventralis); a fruit-piercing stink bug (Glaucias subpunctatus); a red-striped stink bug (Graphosoma rubrolineatum); a brown marmorated stink bug (Halyomorpha mista); a rice stink bug (Lagynotomus elongates, Oebalus pugnax); a two-spotted stink bug (Perillus bioculatus); a conchuela stink bug (Chlorochroa ligata); a Uhler's stink bug (Chlorochroa uhlerii); a Say's stink bug (Chlorochroa sayi); a brown-winged green stink bug (Plautia stali (Scott)); a boxelder bug (Boisea trivittata (Say)); a species of Banasa dimidiata (Say); a species of B. calva (Say); a species of B. euchlora Stål; a Harlequin bug (Murgantia histrionica); a Kudzu bug [Megacopta cribraria (Fabricius)]; a species of Anasa spp., including A. tristis (DeGeer); a species from the genus Leptoglossus; a species of Lygus spp., including a tarnished plant bug (L. lineolaris), a western tarnished plant bug (L. hesperus), a European tarnished plant bug (L. ruguilipnennis), a green leaf bug (L. lucorum); a species of apple dimpling bug (Campylomma spp.); a species of mosquito bug (Helopeltis spp.); a species of a honeylocust plant bug (Diaphnocoris spp.); a species of green mirid (Creontiades spp.); and a species of potato mirid (Calocoris spp).

A method of controlling insects is disclosed. The method may include placing one or more insect repellent compositions, wherein the placement of the one or more repellent compositions is configured to push an insect in a direction of a location, attracting the insect being pushed by the one or more repellent compositions to the location with an insect attractant composition, and capturing the insect at the location, wherein the insect belongs to the order Heteroptera, the Pentatomoidea superfamily, including stink bugs, and shield bugs; the Superfamily Coreoidea, including squash bugs, or the insect family Miridae, including plant bugs.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagrammatical illustration representing a “push-pull” method of insect control;

FIG. 2 is a bar graph illustrating mean numbers of BMSB nymphs captured in the Rescue® stink bug traps baited with either attractants alone or attractants plus different essential oils [as well as a synthetic mixture of Spined Soldier Bug (SBB) aggregation pheromone], Maryland, USA; Jul. 14-Aug. 15, 2011 [bars with the same letter were not statistically different (P>0.05) by Duncan's multiple-range test after ANOVA on log (X+1) transformed data];

FIG. 3 is a bar graph illustrating mean numbers of BMSB adults captured in the Rescue® stink bug traps baited with either attractants alone or attractants plus different essential oils [as well as a synthetic mixture of Spined Soldier Bug (SBB) aggregation pheromone], Maryland, USA; Jul. 14-Aug. 15, 2011 [bars with the same letter were not statistically different (P>0.05) by Duncan's multiple-range test after ANOVA on log(X+1) transformed data];

FIG. 4 is an illustration of simultaneously recorded GC-flame ionization detector (FID) and electroantennographic detector (EAD) (GC-EAD) responses of an BMSB adult antenna to a Solid-Phase Micro-Extraction (SPME) sample of lemongrass essential oil (CAR/PDMS sampling of 1.0 g essential oil in a 20 ml glass vial for 30 sec);

FIG. 5 is an illustration of GC-EAD responses of an BMSB adult antenna to a SPME sample of spearmint essential oil (CAR/PDMS sampling of 1.0 g essential oil in a 20 ml glass vial for 30 sec);

FIG. 6 is an illustration of GC-EAD responses of an BMSB adult antenna to a SPME sample of clove essential oil (CAR/PDMS sampling of 1.0 g essential oil in a 20 ml glass vial for 30 sec);

FIG. 7 is an illustration of GC-EAD responses of an BMSB adult antenna to a SPME sample of ylang ylang essential oil (CAR/PDMS sampling of 1.0 g essential oil in a 20 ml glass vial for 30 sec);

FIG. 8 is an illustration of GC-EAD responses of an BMSB adult antenna to a SPME sample of rosemary essential oil (CAR/PDMS sampling of 1.0 g essential oil in a 20 ml glass vial for 30 sec);

FIG. 9 is an illustration of GC-EAD responses of an BMSB adult antenna to a SPME sample of wintergreen essential oil (CAR/PDMS sampling of 1.0 g essential oil in a 20 ml glass vial for 30 sec);

FIG. 10 is an illustration of GC-EAD responses of an BMSB adult antenna to a SPME sample of geranium essential oil (CAR/PDMS sampling of 1.0 g essential oil in a 20 ml glass vial for 30 sec);

FIG. 11 is an illustration of GC-EAD responses of an BMSB adult antenna to a SPME sample of pennyroyal essential oil (CAR/PDMS sampling of 1.0 g essential oil in a 20 ml glass vial for 30 sec);

FIG. 12 is an illustration of GC-EAD responses of an BMSB adult antenna to a synthetic mixture of the Spined soldier bug aggregation pheromone components (trans-2-hexenal, benzyl alcohol and α-terpineol);

FIG. 13A shows a front view of a first embodiment of a stick pack;

FIG. 13B shows a side view of the stick pack shown in FIG. 13A;

FIG. 13C shows an end view of the stick pack shown in FIG. 13A;

FIG. 14A shows schematically a cross section of the stick pack through section 14-14 in FIG. 13A, showing a solid particulate composition therein;

FIG. 14B shows schematically a cross section of the stick pack through section 14-14 in FIG. 13A, showing a liquid composition therein;

FIG. 15 shows a fragmentary cross-sectional view of a sheet material for stick packs, wherein the various dimensions are exaggerated to illustrate aspects of the sheet material;

FIG. 16 shows schematically a system diagram for an apparatus for producing stick packs packaged with a semiochemical, such as a repellent;

FIG. 17 is a flow diagram illustrating an exemplary method for controlling the rate of release of volatiles of semiochemical compositions, including repellents;

FIG. 18 illustrates another embodiment of a multi-compartment stick pack;

FIG. 19 illustrates another embodiment of a stick pack, having a window portion for the controlled release of volatiles;

FIGS. 20A and 20B illustrate a panel for forming another embodiment of a stick pack, wherein FIG. 20B is a sectional view through section 20B-20B in FIG. 20A;

FIG. 21 is a front view of the stick pack formed from the panel shown in FIGS. 20A and 20B;

FIGS. 22A and 22B show alternative designs for a stick pack similar to the stick pack shown in FIG. 21, with different window configuration; and

FIG. 23 is a perspective view of a trap.

### DETAILED DESCRIPTION

Plant essential oils are one of the major types of botanical products used for insect control. These oils are major sources of highly active and potent metabolites with strong impacts on insect biology, behavior, and physiology. In addition, essential oils have low environmental persistence and mammalian toxicity. More relevantly, they are normally available in large quantities at reasonable prices due to their widespread use as fragrance and food flavors. Essential oils are typically derived by steam distillation from many plant families. They mainly include complex blends of hydrocarbons (monoterpenes and sequiterpenes) and oxygenated compounds such as alcohols, esters, ethers, aldehydes, ketones, lactones, phenols, phenol ethers, and alkaloids. Many essential oils have been shown to have high repellency against various biting insects/arthropods (such as mosquitoes, sand flies, ticks, and other health related pests), stinging insects (such as yellowjackets, paper wasps and hornets; see U.S. Application Publication No. 2012/0107428, filed on Aug. 9, 2011, and several agricultural pests (such as the green peach aphid, Myzus persicae; onion aphid, Neotoxoptera formosana; maize weevil, Sitophilus zeamais; red flour beetle, Tribolium castaneum; two spotted spider mite, Tetranychus urticae; Resselivella oculiperda; and Japanese beetle, Popillia japonica Newman). Owing to their aromatic properties, essential oils would also be a good source of natural repellents for various stink bugs. Furthermore, combinations of repellents (and their release devices) with known attractants (and traps) in a “push-pull” fashion may strengthen the capacity to combat the serious agricultural and home/garden problems caused by the pestiferous stink bugs.

The present application describes field tests of the potential repellency of some representative essential oils against stink bugs using pheromone-baited traps and identifies antennally active compounds from the behaviorally repellent essential oils, which compounds are likely responsible for the repellency, using headspace sampling (SPME), gas chromatographic (GC)-electroantennographic detection (EAD), and GC-mass spectrometry (MS), determines behavioral activity (potential repellency) of some EAD-active compounds in the field using attractant-baited traps, discloses a significant repellency effect of the synthetic aggregation pheromone of a predator bug, the Spined soldier bug, on the BMSBs, and BMSB antennal responses to these predator pheromone components.

In some embodiments, employment of a repellent composition and a controlled release device provides repellency. Such devices are described herein, and it is noted that selection of a device may enhance the effects of the repellent composition, as adjusting and metering the concentration of the volatilized repellent composition per cubic meter of air helps to achieve maximum repellency. Factors affecting device selection include the insect targeted, time of year (as temperature correlates to a volatile's release rate), environment (e.g., closed space, open space, calm day, windy day, low humidity environment (e.g., Arizona), and high humidity environment (e.g., Florida)). Use of a controlled release device in combination with a repellent composition may provide repellency effects that are greater than use of the repellent composition alone. Repellent compositions are suitable to be used both indoors and outdoors.

In one embodiment, the present disclosure describes a method for controlling insects. Controlling insects, as used herein, means to change a behavior of the insect, such as to cause the insect to be repelled, for example. A method may include releasing into a space an essential oil or a mixture of essential oils selected from clove oil, lemongrass oil, spearmint oil, ylang ylang oil, wintergreen oil, rosemary oil, geranium oil, pennyroyal oil, or any combination thereof. In one embodiment, the method includes repelling from a space or a surface one or more insects selected from any insects of the order Heteroptera, and the superfamily Pentatomoidea, the Superfamily Coreoidea, and the family Miridae, commonly referred to as stink bugs, squash bugs, and plant bugs, respectively. Any of the methods disclosed herein may be practiced both indoors and outdoors.

In another embodiment, a method includes releasing into a space a synthetic compound or a mixture of compounds selected from I-menthone, P-menthone, eugenol, E-citral, Z-citral, pulegone, methyl benzoate, l-carvone, methyl salicylate, benzyl acetate, β-caryophyllene, geraniol, or any combination thereof. These compounds were identified from the repellent essential oils disclosed above and show significant antennal responses by stink bug species that are largely responsible for the repellency of the essential oils. Any synthetic isomer of the above disclosed compounds may also be used as a repellent, such as a skeletal isomer or a position isomer, or a stereoisomer. A method may include repelling from a space or a surface one or more insects selected from any insects of the order Heteroptera, and the superfamily Pentatomoidea, the Superfamily Coreoidea, and the family Miridae, commonly referred to as stink bugs, squash bugs, and plant bugs, respectively. It should also be appreciated that any essential oil not specifically listed above, but that contains any of the identified compounds recited above, is included within the scope of this disclosure. Methods of identifying whether a compound is a major constituent in an essential oil are well-known in the art.

In another embodiment, a method for controlling insects is disclosed. The method includes releasing into a space any other essential oil(s) not listed above but containing any identified repellent compounds disclosed herein as a major constituent(s).

In another embodiment, a method includes releasing into a space a synthetic mixture of the Spined soldier bug aggregation pheromone components (trans-2-hexenal, benzyl alcohol and α-terpineol). A method may include repelling from a space or a surface one or more insects selected from any insects of the order Heteroptera, and the superfamily Pentatomoidea, the Superfamily Coreoidea, and the family Miridae, commonly referred to as stink bugs, squash bugs, and plant bugs, respectively.

In other circumstances, stink bugs overwinter inside of human structures, such as attics, living rooms, and industrial buildings. Accordingly, it would be desirable to repel insects from certain spaces or surfaces; especially to prevent stink bugs from entering the homes. The present disclosure can advantageously provide such protection.

Space as used herein refers to any areas, including air space, ground space, water space, and in or on things, plants, trees, or structures.

The essential oils and compounds of the disclosed methods are volatile, and, thus, one mode of application is to provide the essential oils or compounds in a form that is freely volatile. However, it is within the scope of this disclosure to use the compounds in a manner such that they may be bound to materials that reduce the volatility and slowly release the active repellent compositions.

The repellent essential oils and compounds disclosed herein are readily available from many commercial chemical supply stores. Synthetic, when used to describe the compounds, means that compounds were purified from a natural source, manufactured, or synthesized. Natural sources of these compounds include, but are not limited to, the essential oils: clove oil (Myrtaceae), lemongrass oil (Poaceae), spearmint oil (Lamiaceae), ylang ylang oil (Annonaceae), wintergreen oil (Ericaceae), rosemary oil (Lamiaceae), geranium oil (Geraniaceae), and pennyroyal oil (Lamiaceae). Methods of isolating these compounds from natural sources are well-known in the art.

The behaviorally repellent essential oils (individually or in combinations) or their antennally-active and behaviorally repellent synthetic compounds and their isomers may be formulated alone or with other ingredients and released at an effective amount from suitable devices or dispensers to provide a spatial attraction-inhibitor/repellent composition for controlling or repelling various species of pestiferous stink bugs. The essential oils or compounds alone may provide repellency against the insects. The application of the essential oils or compounds for spatial attraction-inhibition/deterrence/repellency include, but are not limited to the following methods. For use as a spatial repellent or attraction-inhibitor, the methods may include releasing into a space where one wants to provide such inhibiting or deterring or repelling effect or dispensing or applying onto a protection device, such as a dispenser for a target, at least one spatial inhibitor/repellent essential oil selected from the following essential oils: clove oil, lemongrass oil, spearmint oil, ylang ylang oil, wintergreen oil, rosemary oil, geranium oil, pennyroyal oil, or any combination thereof, or at least one compound from the following antennally or behaviorally active compounds, such as I-menthone, P-menthone, eugenol, E-citral, Z-citral, pulegone, methyl benzoate, l-carvone, methyl salicylate, benzyl acetate, β-caryophyllene, geraniol, or any combination thereof. The method may also include releasing into the space or surface any other essential oil(s) not listed but containing any identified repellent compound(s) as a major constituent. The method may also include releasing into the space or surface a synthetic mixture of the Spined soldier bug aggregation pheromone components (trans-2-hexenal, benzyl alcohol and α-terpineol).

As used herein, “stink bugs” are insects of the Pentatomoidea superfamily, which include stink bugs, and shield bugs. “Squash bugs” are insects of the Superfamily Coreoidea, and the “plant bugs” are insects of the family Miridae. Any one or more of these stink bugs, squash bugs or plant bugs disclosed herein may be repelled by the disclosed repellent compositions. A variety of stink/squash/plant bugs are known in the art. Non-limiting examples of stink bugs include the green stink bug (Acrosternum hilare (Say)); several species of Euschistus spp. such as the brown stink bug (Euschistus servus (Say)), E. tristigmus, E. conspersus, E. variolarius, E. politus, or E. heros; the southern green stink bug (Nezara viridula (L.)); the eastern green stink bug (Nezara antennata); the Spined soldier bug (Podisus maculiventris); the East Asian stink bug or the yellow-brown stink bug or the brown marmorated stink bug (Halyomorpha halys); the red-shouldered stink bug (Thyanta pallidovirens); the globular stink bug (Megacopta punctatissimum); the white-spotted stink bug (Eysarcoris ventralis); the fruit-piercing stink bug (Glaucias subpunctatus); the red-striped stink bug (Graphosoma rubrolineatum); the brown marmorated stink bug (Halyomorpha mista); the rice stink bug (Lagynotomus elongates, Oebalus pugnax); the two-spotted stink bug (Perillus bioculatus); the conchuela stink bug (Chlorochroa ligata); Uhler's stink bug (Chlorochroa uhlerii); Say's stink bug (Chlorochroa sayi); the brown-winged green stink bug (Plautia stali (Scott)); the boxelder bug (Boisea trivittata (Say)); Banasa dimidiata (Say); B. calva (Say); B. euchlora Stål; the Harlequin bug (Murgantia histrionica); and the Kudzu bug [Megacopta cribraria (Fabricius)]. Non-limiting examples of squash bugs include: Anasa spp., especially the well-known A. tristis (DeGeer); and many species from the genus Leptoglossus. Non-limiting examples of plant bugs include: Lygus spp, such as the tarnished plant bug (L. lineolaris), the western tarnished plant bug (L. hesperus), the European tarnished plant bug (L. ruguilipnennis), and the green leaf bug (L. lucorum); the apple dimpling bugs (Campylomma spp.); the mosquito bugs (Helopeltis spp.); the honeylocust plant bugs (Diaphnocoris spp.); the green mirids (Creontiades spp.); and the potato mirids (Calocoris spp).

As used herein, an “effective amount” of an essential oil or any essential oil compound is an amount effective to achieve a desired result, such as to prevent a stink bug from reaching a location. The amount of essential oil and/or compound that is used alone or in a composition may be determined experimentally.

Any essential oil or synthetic compound may comprise about, at most about, or at least about a weight percent of 0.1, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.6, 97, 97.5, 98, 98.5, 99, or 99.5% or more, but less than 100%, of a composition.

In any embodiment herein, a composition may comprise a synergistic amount of one or more essential oils, one or more synthetic compounds, or any combination thereof, to provide a synergistic repellency, deterrent, or attraction-inhibitory effect. As used herein, a “synergistic amount” refers to an amount that produces greater than additive effects.

Any embodiment herein may comprise, consist essentially of, or consist of components, ingredients, steps, etc. For example, any composition may consist or consist essentially of any essential oil or synthetic compound. With respect to “consist essentially of,” such embodiments are drawn to the specified components, ingredients, steps, etc., and those that do not materially affect the basic and novel characteristics of the claimed invention. Non-limiting examples of those that do not materially affect the basic and novel characteristics of the claimed invention include antioxidants (e.g., butylated hydroxytoluene, or BHT) and vegetable oils as inert controlled release agents, fillers, fragrances, diluents, and the like. With respect to “consist of,” such embodiments are drawn to the specified components only.

The dispensing of such natural essential oils or synthetic compounds may be by way of evaporation or volatilization of the active essential oils or compounds from a device with either a controlled release or a passive release method, such as dispersion by an aerosol or powder that can be scattered on the ground, and the like.

There can also be provided a controlled release device that is used to control the release rate of volatilization of the essential oil(s) or the synthetic compound(s). A release device can be a container having a space therein to house a material onto which one or more of the essential oils or one or more of the compounds is impregnated. The material has sufficient free void space to take in or absorb a quantity of essential oils or compounds sufficient to achieve a desired effect (e.g., repellency). Suitable materials can be fibrous, porous, solids, or flexible materials. Suitable materials may include absorbent materials such as paper, porous plastics, absorbent minerals, carbon, and the like. The release device can have an opening on the outer surface thereof to permit the vapors emitted from the essential oil or oils or compounds to escape the device. Preferably, the device includes means for closing the opening, such as when the device is not in use, and, more preferably, the size of the opening can be made adjustable to allow the user of the device control over whether to emit more or less of the vapors, including complete shut off. The release device can vary in its shape or size to accommodate short periods of efficacy or long periods of efficacy. Devices can come in sizes made to last days or weeks by altering the amount of essential oils and compounds that are loaded into the absorbent material.

The material within the device can be replaced with a new material when the essential oils or synthetic compounds have been depleted. To this end, absorbent materials may come preloaded with the essential oils and compounds and made separately available to be placed in the device by the user. Additionally, the device can be made to accept differing sizes of the absorbent material to allow selecting short or long periods of efficacy. Materials having different essential oils or compounds can be preloaded and made available to a user, such that some essential oils or compounds can be more effective toward one kind of insect. The package may indicate which insect is repelled so that the user is able to change materials, but is only required to purchase one device. This permits a user to tailor the device to a particular length of use and for a particular insect. The devices can be made from plastics or other suitable materials of construction. Devices can be injection molded. Other forms of release devices made for the home or an exterior location can vary in their shape or size to fit different settings, such as incorporating the compounds into ornaments to be inconspicuously placed in indoor or outdoor locations, or can be used for dual purposes, such as decorations having insect-repellent properties, and for use in buildings.

Furthermore, these repellent devices or dispensing formulations (solids—powders, impregnated plastics, impregnated fibers; or liquids—sprays, aerosols; or gels—creams) can be easily applied to prevent insects from approaching targets both indoors and outdoors, that is, to outdoor settings such as the eaves of building structures, walls and windows, vegetable plants in the gardens and fruit trees in the yards or orchards, etc. Such repellent formulations can also be deployed in cooperation with pheromone attractant-baited traps (set up around the target plants or trees to pull the stink bugs away from the targets) for these stink bugs in a push-pull tactic, which would likely enhance the pest control efficacy against these pestiferous stink bugs.

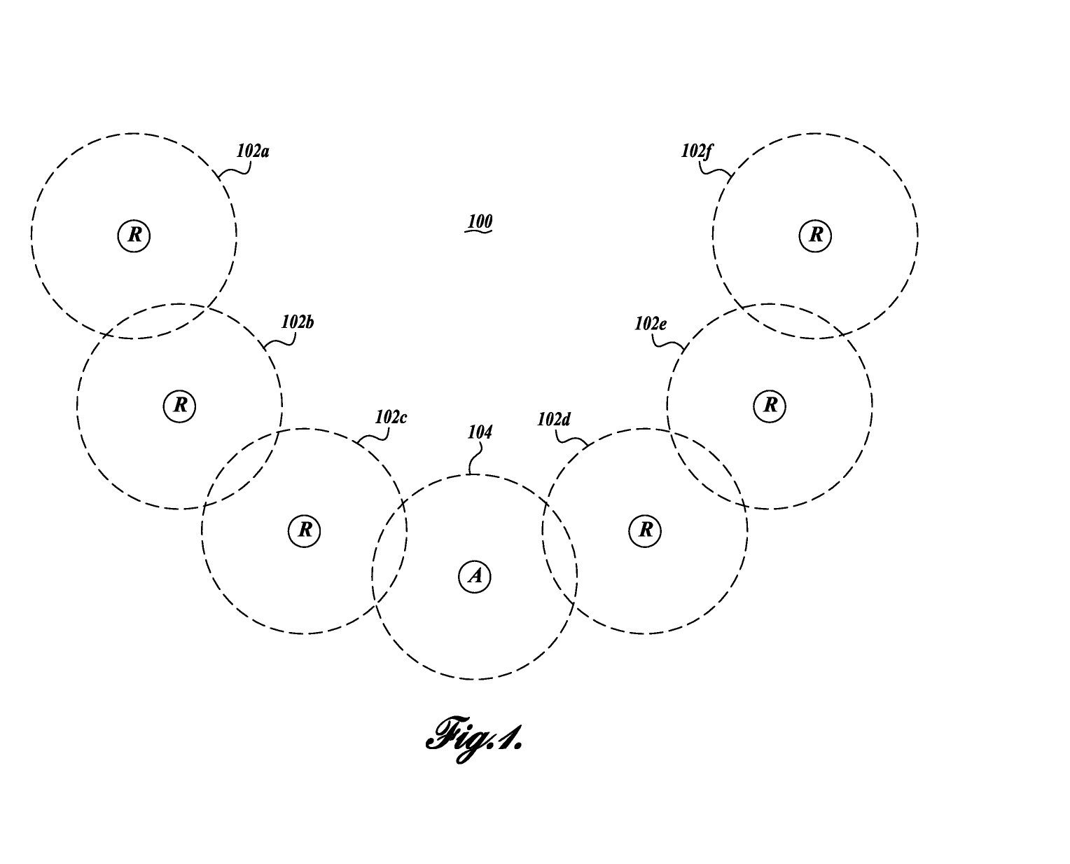

As used herein, “push-pull” is a method of insect control combining the use of repellents and attractants. Generally, a “push-pull” method can include placing one or more insect repellent compositions, wherein the placement of the one or more repellent compositions is configured to push an insect in a specific direction to a designated location where an attractant composition is located. The methond includes attracting the insect being pushed by the repellent compositions to the location with an insect attractant composition. The method may include capturing the insect at the location with a trap. The “push-pull” method is effective to control an insect which belongs to the order Heteroptera, the Pentatomoidea superfamily, including stink bugs, and shield bugs; the Superfamily Coreoidea, including squash bugs, or the insect family Miridae, including plant bugs. A non-limiting example of a “push-pull” method is illustrated in FIG. 1. In a push-pull method of insect control, one or more sites with repellents “R” 102a-f can be provided to define a perimeter of repellency that insects do not approach. Each repellent site may itself define a radius of repellency which insects do not approach. Placing repellents so that each radius overlaps with the adjacent radius creates a continuous perimeter of repellency. Once the insect enters the general area 100, the insect is contained within the repellent perimeter, but may escape the same way from which it entered. If the insect continues to travel forward, the insect will be repelled by the repellents within the perimeter on the left and on the right, leading the insect to the attractant 104. In this manner, the insect can be pushed in the direction of the attractant “A” 104. Once the insect enters the radius within which the attractant is effective, the insect will be pulled toward the center of the attractant site 104. The center of the attractant site 104 may additionally contain a trap to capture the insect. In the embodiment shown, the repellent sites 102a-f are configured to funnel the insect toward the attractant site 104. However, in other embodiments, the repellent sites may be configured differently, and may take advantage of physical structures, such as walls, to lead or “push” the insect in the direction of the attractant.

As the repellent to be used in the above-described push-pull method, any one or more of the repellents disclosed herein may be used. As the attractant to be used in the above-described push-pull method, any one or more of the attractants disclosed in applicants' U.S. application Ser. No. 13/180,281, incorporated herein expressly by reference, may be used.

Aggregation pheromone components have been identified from several agriculturally important stink bugs. For instance, methyl (2E,4Z)-decadienoate (M24DD) has been identified as an aggregation pheromone component or field attractant for seven Euschistus spp. and has been used for stink bug monitoring programs in agricultural settings. Another methyl ester, methyl (2E,4E,6Z)-decatrienoate (M246DT), was identified as an aggregation pheromone component of the stink bug Plautia stali Scott in Japan and recently as a field attractant for both adults (males and females) and nymphs of the BMSB, Halyomorpha halys (Stål) and A. hilare (Aldrich et al., J. Chem. Ecol. 33:801 (2007)); Khrimian, Tetrahedron 61:3651 (2005)).

A sesquiterpene epoxyalcohol, mumantiol, was identified as an aggregation pheromone component of the Harlequin bug, Murgantia histrionica (Hahn) (Zahn et al., J. Chem. Ecol. 34:238 (2008)). As reported by Zahn et al, sexually mature male Harlequin bugs produce a sex-specific compound, identified as one of the stereoisomers of the sesquiterpene epoxyalcohol 4-[3-(3,3-dimethyloxiran-2-yl)-1-methylpropyl]-1-methylcyclohex-2-en-1-ol (murgantiol).

Murgantiol, the reported pheromone for the Harlequin bug, was recently found to be an indoor attractant for stink bugs, such as both sexes of BMSB adults during the overwintering and transition periods (U.S. application Ser. No. 13/180,281, filed Jul. 11, 2011), and as an outdoor attractant for the BMSBs during the summer mating/oviposition and feeding season (U.S. application Ser. No. 13/410,124, filed Mar. 1, 2012). Further, murgantiol together with stink bug attractants such as M246DT and M24DD were found to synergistically attract BMSB and Euschistus spp. Accordingly, murgantiol may be used both indoors and outdoors to attract stink bugs, such as BMSBs, in the push-pull method. Furthermore, murgantiol along with other known attractants, can be used to attract stink bugs, such as BMSBs, in the push-pull method.

Murgantiol may be obtained synthetically as described by Zahn et al., J. Chem. Ecol. 34:238 (2008). Murgantiol is a compound with four chiral centers and 16 possible stereoisomers. The relative and absolute configurations of the insect-produced compound were not identified by Zahn et al. and are presently unknown, but the structure was reported as follows, with carbon numbering shown:

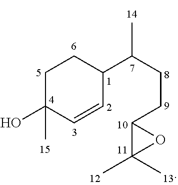 [SMILES: CC(CCC1OC1(C)C)C1C=CC(C)(O)CC1]

As used herein, “murgantiol” refers to any compound or mixture of compounds (isomers) that exhibits the murgantiol skeleton structure noted above. Thus, murgantiol may refer to a single isomer, a mixture of all 16 isomers (racemic murgantiol), or a mixture of some of these isomers (e.g., a mixture of 2, 3, 4, 5, 6, 7, 8, 9, 10, 11, 12, 13, 14, or 15 isomers, or a mixture of at most or at least 2, 3, 4, 5, 6, 7, 8, 9, 10, 11, 12, 13, 14, or 15 isomers). A mixture may comprise a racemic mixture. A mixture may, for example, entail a mixture of more polar isomers, such as the 4R/S-isomers (4R/S-1S-7S-10S-isomer, 4R/S-1R-7S-10S-isomer, 4R/S-1R-7R-10S-isomer, 4R/S-1R-7R-10R-isomer, 4R/S-1S-7R-10R-isomer, 4R/S-1S-7R-10S-isomer, 4R/S-1S-7S-10R-isomer, and the 4R/S-1R-7S-10R-isomer). A mixture may, for example, entail a mixture of less polar isomers, such as the 4S/R-isomers (4S/R-1S-7S-10S-isomer, 4S/R-1R-7S-10S-isomer, 4S/R-1R-7R-10S-isomer, 4S/R-1R-7R-10R-isomer, 4S/R-1S-7R-10R-isomer, 4S/R-1S-7R-10S-isomer, 4S/R-1S-7S-10R-isomer, and the 4S/R-1R-7S-10R-isomer). Some mixtures may entail four isomers, such as the 4R/S-7R/S-isomers (4R/S-7R/S-1R-10R-isomer, 4R/S-7R/S-1R-10S-isomer, 4R/S-7R/S-1S-10S-isomer, and the 4R/S-7R/S-1S-10R-isomer); the 4R/S-7S/R-isomers (4R/S-7S/R-1R-10R-isomer, 4R/S-7S/R-1R-10S-isomer, 4R/S-7S/R-1S-10S-isomer, and the 4R/S-7S/R-1S-10R-isomer); the 4S/R-7R/S-isomers (4S/R-7R/S-1R-10R-isomer, 4S/R-7R/S-1R-10S-isomer, 4S/R-7R/S-1S-10S-isomer, and the 4S/R-7R/S-1S-10R-isomer); and the 4S/R-7S/R-isomers (4S/R-7S/R-1R-10R-isomer, 4S/R-7S/R-1R-10S-isomer, 4S/R-7S/R-1S-10S-isomer, and the 4S/R-7S/R-1S-10R-isomer). In some embodiments, murgantiol is further defined as a mixture of diastereomers of 4-[3S-(3,3-dimethyloxiran-2-yl)-1-methylpropyl]-1-methylcclohex-2-en-1-ol. In some embodiments, murgantiol is further defined as a mixture of diastereomers of 4-[3R-(3,3-dimethyloxiran-2-yl)-1-methylpropyl]-1-methylcyclohex-2-en-1-ol.

In some embodiments, a composition comprises an isolated mixture defined as a more polar mixture of murgantiol stereoisomers obtained and separated from a racemic mixture, such as a more polar mixture of murgantiol stereoisomers (4R/S-isomers). In some embodiments, a composition comprises an isolated mixture defined as a less polar mixture of murgantiol stereoisomers obtained and separated from a racemic mixture, such as a less polar mixture of murgantiol stereoisomers (4S/R-isomers). The “more polar mixture” and the “less polar mixture” can be obtained and separated from a racemic mixture of murgantiol by flash chromatography on silica gel, eluting with 10% ethyl acetate in hexane, with the first fraction being the less polar murgantiol mixture and the later eluting fraction being the more polar murgantiol mixture. Each fraction constains 8 isomers that are inseparable by the flash chromatography. As noted herein, a composition may comprise either the 8 less polar isomers or the 8 more polar isomers. In some embodiments, a composition comprises a racemic mixture of murgantiol stereoisomers (all 16 stereoisomers).

As used herein, an “effective amount” of murgantiol is an amount effective to achieve a desired result, such as to attract a stink bug to either an indoor or outdoor predetermined location, such as a trap that is outdoors. The amount of murgantiol that is used alone or in a composition may be determined experimentally, but ranges of 0.1 to 100 mg may be suitable. For example, at least or at most 0.1, 10, 20, 30, 40, 50 60, 70, 80, 90, or 100 mg of murgantiol may be employed, or any range derivable therein. Murgantiol may be used alone or in a composition, such as in a composition further comprising one or more semiochemicals, oils, antioxidants, or other additives, or any combination thereof.

As used herein, a “synergistic amount” refers to an amount that produces greater than additive effects. A “synergistic composition” is a composition comprising a synergistic amount of two or more components, typically murgantiol plus at least an additional stink bug attractant. A “murgantiol-containing synergistic composition” comprises a synergistic amount of a first stink bug attractant that is murgantiol and at least a second stink bug attractant.

Commonly known stink bug attractants include methyl (2E,4Z)-decadienoate (M246DT) and methyl (2E,4Z)-decadienoate (M24DD), and stereoisomers thereof. Additional semiochemicals that may be included in a composition with murgantiol include M246DT, methyl (2E,4Z,6Z)-decatrienoate, methyl (2E,4E,6E)-decatrienoate, methyl (2Z,4E,6Z)-decatrienoate, or other stereoisomers; M24DD; ethyl (2E,4Z)-decadienoate; methyl (2Z,4E)-decadienoate; and methyl (2E,4E)-decadienoate; or any combination thereof.

An attractant composition may comprise murgantiol. An attractant composition may comprise a synergistic amount of a first stink bug attractant that is murgantiol and at least a second stink bug attractant. In some embodiments, the second stink bug attractant is M246DT. In some embodiments, the second stink bug attractant is M24DD. An attractant composition may further comprise a third stink bug attractant. In some embodiments, the second stink bug attractant is M246DT and the third stink bug attractant is M24DD.

An attractant composition may consist essentially of a synergistic amount of a first stink bug attractant that is murgantiol and at least a second stink bug attractant. Such a composition includes the specified components and those that do not materially affect the basic and novel characteristics of the composition. For example, antioxidants, oils, and impurities may be comprised in such compositions.

Also provided is a composition consisting of a synergistic amount of a first stink bug attractant that is murgantiol and at least a second stink bug attractant. Such a composition excludes other components.

In some embodiments, the only stink bug attractants are murgantiol and a second stink bug attractant, such as M246DT or M24DD. In some embodiments, the only stink bug attractants are murgantiol, M246DT, and M24DD.

In some embodiments, the attractant composition comprises an isolated mixture defined as a more polar mixture of murgantiol stereoisomers obtained and separated from a racemic mixture; and at least one of M246DT and M24DD, and their related stereoisomers, or a combination thereof. In some embodiments, the attractant composition comprises an isolated mixture defined as a less polar mixture of murgantiol stereoisomers obtained and separated from a racemic mixture; and at least one of M246DT and M24DD, and their related stereoisomers, or a combination thereof. An attractant composition may comprise a racemic mixture of murgantiol stereoisomers; and at least one of M246DT and M24DD, and their related stereoisomers, or a combination thereof. Both repellent and attractant compositions may be comprised in separate devices or dispensers that controllably releases the composition.

Any device suited for the purpose may be used in the controlled release of repellents. A particular embodiment of a controlled release device for use to release repellents is a stick pack.

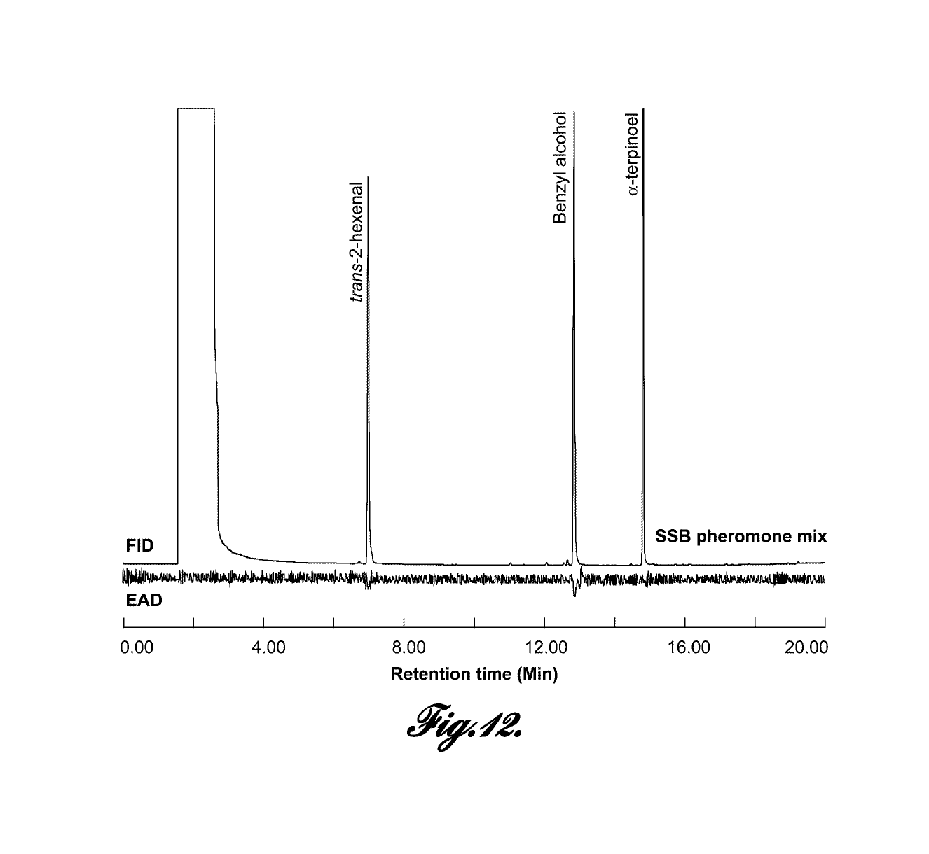

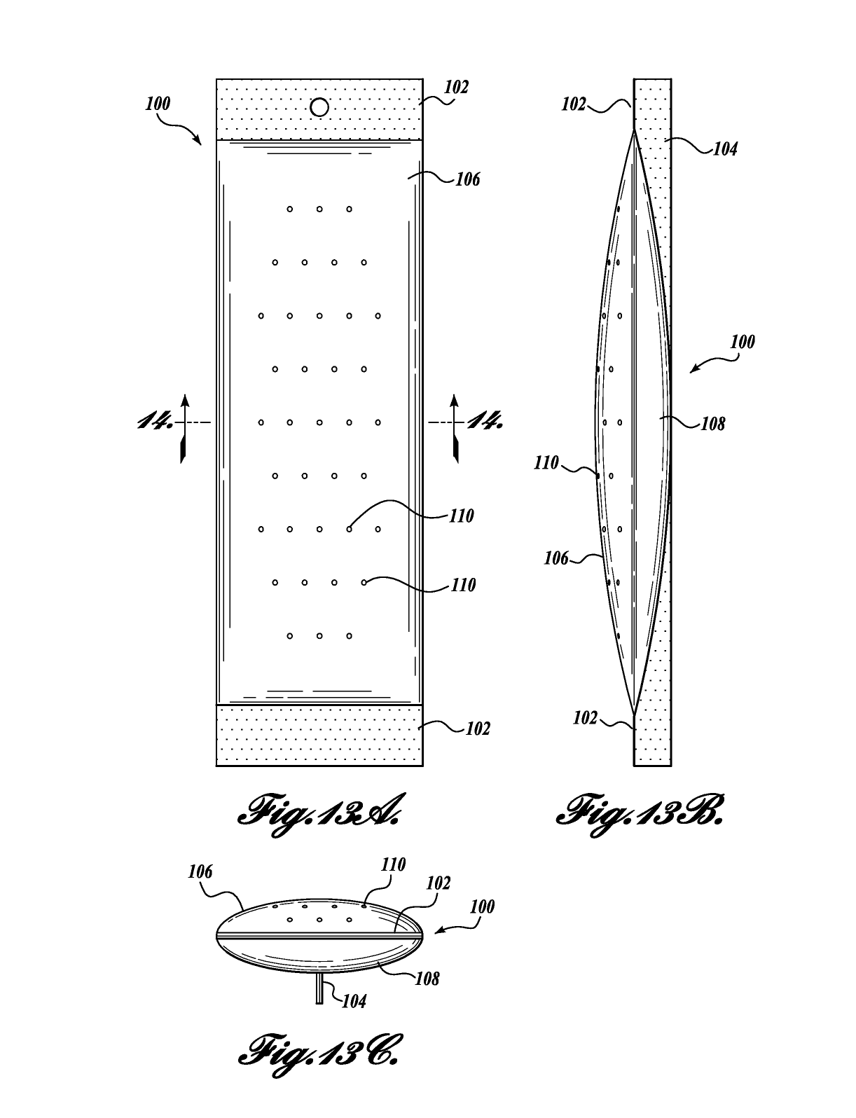

FIGS. 13A, 13B, and 13C illustrate front, side, and end views, respectively, of a sachet or stick pack 100. The stick pack 100 is a generally tubular structure formed from a sheet of material, preferably a polymeric sheet comprising multiple layers or laminae. The end portions 102 are sealed transversely, and a longitudinal sealed portion 104 closes the tubular structure, such that a volume is defined between the ends 102.

As discussed below, the properties and configuration of the multiple layers for stick packs 100 cooperatively restrict and control the release rate of volatiles from the composition that is packaged in the stick pack 100. In particular, the designer may select the materials and certain characteristics of the layers used for the sheet of material to achieve a desired volatile release rate. For example, the layer material properties (e.g., the porosity of the material to the selected composition volatiles), the thickness of the layers, the characteristics of optional apertures (e.g., number, density, size, depth, and shape).

In the embodiment of FIGS. 13A-13C, a front panel 106 of the stick pack 100 optionally includes a pattern of micro-perforations 110 that are sized and configured to achieve a desired release rate of volatiles, as discussed below. In the current embodiment, a back panel 108 portion of the stick pack 100 does not include any micro-perforations. However, it is contemplated that in some applications it will be desirable that the back panel 108 also include micro-perforations 110. In some applications, the stick pack may be formed from a suitably permeable or porous material, wherein the permeability is sufficient to achieve a desired volatile release rate without the use of micro-perforations.

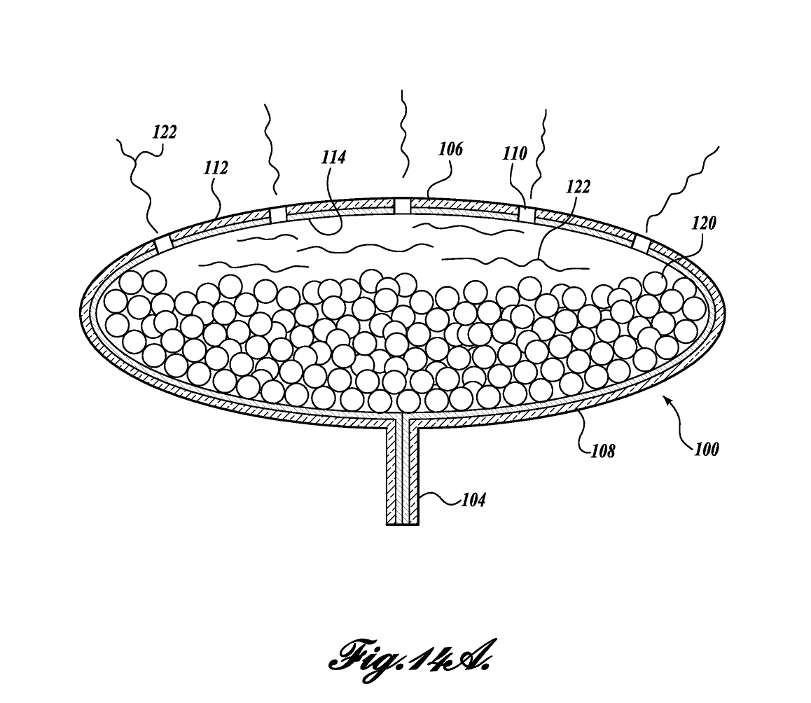

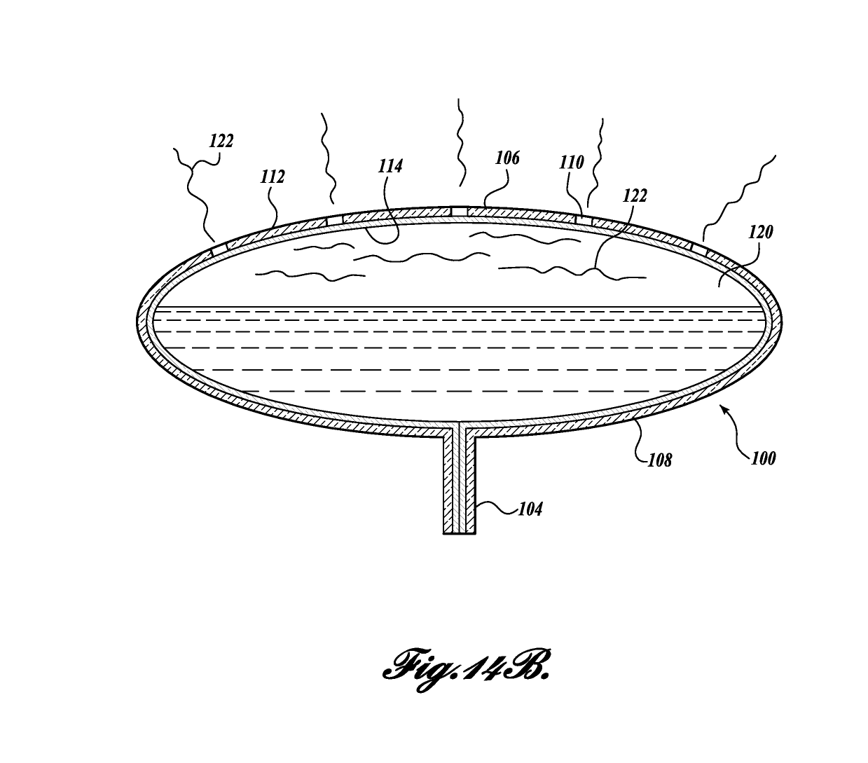

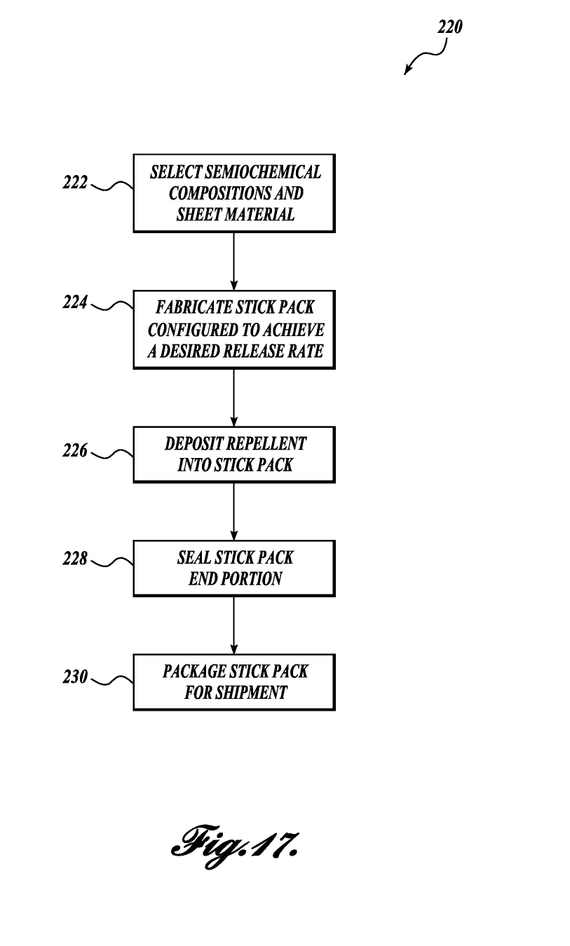

FIG. 14A illustrates a cross section of the stick pack 100 with the composition 120 in the stick pack 100 illustrated generically. In this exemplary embodiment, the innermost layer 114 comprises a material having a relatively low density that is suitable for heat welding to form effective seals. The innermost layer 114 may also be selected for its permeability to the composition volatiles 122. The outermost layer 112 is bonded or otherwise adhered to the innermost layer 114 and is formed of a relatively higher density material selected for its barrier functionality, mechanical strength, dimensional stability, and suitability for manipulation in a high speed stick pack machine (see, FIG. 16). The composition 120 may include one or more essential oils, their constituent compounds, and other components that may be desired, for example, to stabilize or otherwise affect the chemical or mechanical properties of the composition 120.

In FIG. 14A, the composition 120 is illustrated in an idealized bead, powder, or particulate form having a characteristic size or dimension (e.g., diameter). Preferably, the micro-perforations 110 are sized to prevent the loss of the particulates therethrough. It is also contemplated that the composition 120 may alternatively be in liquid form, incorporated into a gel, paste, or solid matrix, or absorbed into a porous medium such as a sponge or paper, for example. In liquid form, the composition may be of relatively low viscosity, or a very viscous or viscoelastic material. The selected composition volatilizes at the environmental conditions contemplated for its intended use. The quantity of composition 120 may be such that the volume enclosed by the stick pack 100 is only partially filled by the composition 120. The remaining volume in the stick pack 100 may be partially or substantially filled with vapors or volatiles 122. The volatiles 122 escape or are gradually released through the micro-perforations 110, and/or through any permeable layer defined by the stick pack 100.

In FIG. 14B, the composition 120 is illustrated in an idealized liquid form. If the composition 120 is in liquid form, it is contemplated that the micro-perforations 110 will extend only through the outer layers 112, and the non-perforated inner layer 114 will therefore prevent any leakage of liquid composition 120 therethrough. The inner layer 114, of course, is selected to permit a gradual release of volatiles.

The rate of release of the volatiles 122 will depend in part on the characteristics of the micro-perforations 110. For example, the rate of release may depend on micro-perforation parameters such as (1) the number of perforations; (2) the size or distribution of sizes of the perforations; (3) the spacing and pattern of the perforations; (4) the shape of the perforations (e.g., elongate, star-shaped, circular); (5) the depth of the perforations (e.g., extending partially through the substrate); and (6) any blockage of the perforations. The designer and/or the user, therefore, have a number of parameters that may be used to control the rate of release of volatiles 122.

For example, the designer may select the size and number of micro-perforations 110 to accommodate a particular combination of semiochemicals 120 to achieve a desired release rate. A composition 120 having a semiochemical with a low volatility may require more and larger perforations than one with a semiochemical that is highly volatile. In another example, different configurations of micro-perforations 110 may be available, depending on the anticipated environmental conditions (e.g., temperature, humidity) for the expected use of the composition 120. For example, one configuration of micro-perforations in a stick pack 100 may be suitable when lower temperatures are expected, and a different configuration may be suitable at higher temperatures. A family of stick packs 100 may be made available to users, who will then select the particular stick pack 100 that suits their application. Optionally, a blocking element (not shown), for example, a strip of adhesive, a sleeve, or the like, may be provided to selectively block some portion of the micro-perforations 110, to selectively adjust the rate of release of volatiles 122, for example, to adjust for environmental conditions or to accommodate particular situations.

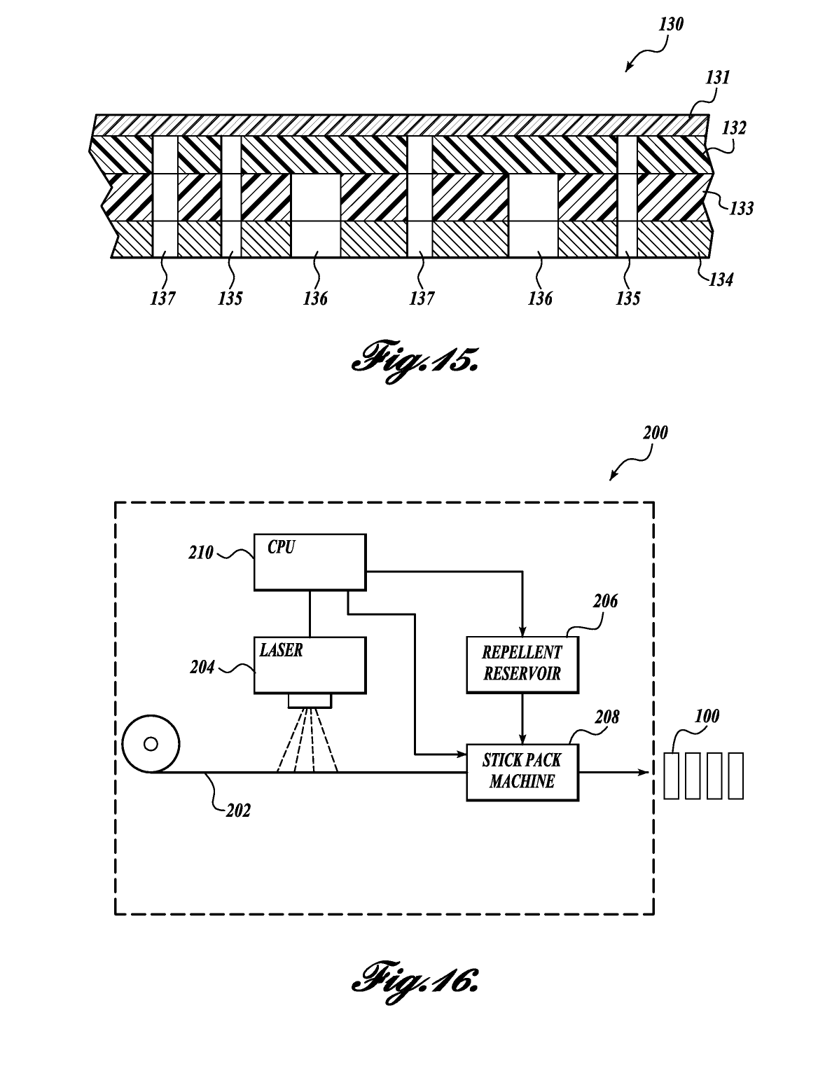

FIG. 15 illustrates an exemplary fragmentary cross section of a sheet 130 that may be used to form the stick pack 100. The sheet 130 includes one or more polymeric laminae, and may additionally include paper or foil laminae (barrier layer), for example. In this exemplary embodiment, the sheet 130 comprises four laminae 131, 132, 133, 134. An exemplary total thickness of the sheet 130 is in the range of 5.0 to 400.0 microns. In a current embodiment, the total thickness is between about 30.0 microns and 300.0 microns. The multiple laminae 131, 132, 133, 134 may be provided to produce a desired release rate of volatiles 122, and to achieve desired mechanical and manufacturability properties. For example, the material for the innermost lamina 131 may be selected, in part, for its ability to produce good and consistent longitudinal and end seals for the stick pack 100.

The material for one or more of the laminae 131, 132, 133, 134 may also be selected based on the permeability of the material to the volatiles, providing an additional parameter to control the release rate of particular volatiles 122.

In FIG. 15, the micro-perforations have varying diameters and varying depths of penetration through the sheet 130. For example, micro-perforations 135 are relatively small in diameter and extend through the outer lamina 134 and all the way to the inner lamina 131. If a solid composition is to be used, for example, the micro-perforations 135 may alternatively extend through the inner lamina 131. Therefore, molecules of suitable size may escape from the stick pack 100 through the apertures 135. Micro-perforations 136, although relatively large in diameter, only extend through the two outermost laminae 133, 134. Therefore, only molecules that are permeable to the innermost laminae 131, 132 will readily escape through these micro-perforations 136. Micro-perforations 137 are of intermediate diameter, and extend through the three outermost laminae 132, 133, 134 in this exemplary embodiment.

Therefore, it will be appreciated that a stick pack 100 may be designed to contain a plurality of different semiochemicals in a mixture or agglomeration, and to provide different release rates for each of the different semiochemicals.

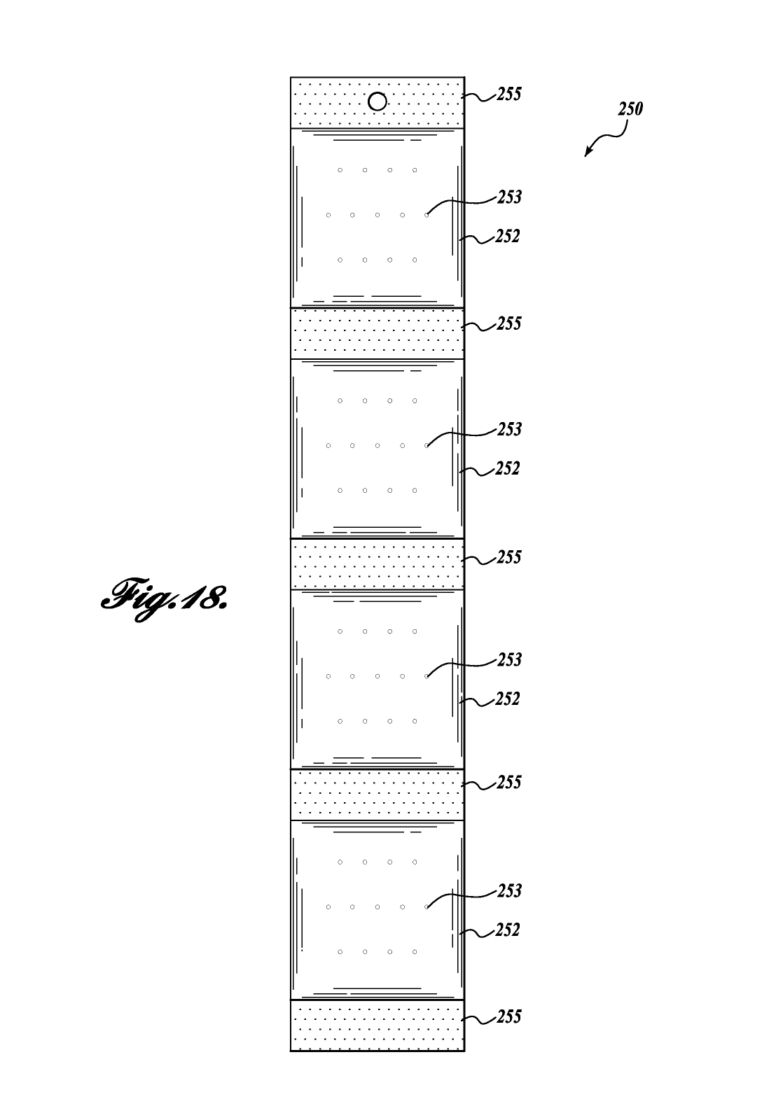

FIG. 17 illustrates a system 200 for producing a stick pack 100. The system 200 in this embodiment takes a roll of sheet material 202 and selectively directs a laser system 204 to produce a desired pattern of micro-perforations in or through the sheet material 202. Different commercial laser systems are suitable. For example, it is known in the packing industry to use CO₂ lasers, such as “sealed off” coherent CO₂ lasers. Such lasers are suitable for use to process paper, plastic film, and other flexible materials. By some accounts, the sealed off coherent CO₂ laser has become a tool of choice to process packaging materials due to its reliability, low cost, compact footprint, and high quality with respect to laser power and beam characteristics.

A reservoir 206 of the desired composition provides product to a stick pack machine 208 that receives the sheet material 202 and forms the final stick pack 100. The operation is controlled with a computer or stand-alone central processing unit (CPU) controller 210 that may be separate or integrated into the stick pack machine 208. The controller 210 is programmable to accommodate different sheet material 202 and composition 120, such that the system 200 may be operated to produce any number of different products.

A simplified flow chart 220 of a method is shown in FIG. 17. The user first selects in block 222 one or more compositions or compounds and sheet material for a particular application. The compositions are selected with reference to the target insect. The selection may include the particular form, including any matrix material that may be useful for stabilizing or controlling the rate of volatilization. The composition may also include components to confer particular aesthetic aspects to the composition, such as color or scent. A composite sheet material for the stick pack package is also selected. The selection of the sheet material 202 may require consideration of the particular composition selected. For example, the innermost lamina of the sheet material must be compatible with the composition. One or more of the laminae may be selected for their permeability with respect to one or more of the compositions.

The packaging for the stick pack 100 is fabricated in block 224, configured for the desired release rate of the volatiles, for example, with micro-perforations and/or selected permeability properties. The selected semiochemical(s) are deposited into the packaging or onto the sheet prior to sealing the package in block 226. The stick pack ends and longitudinal seam are sealed in block 228. The stick pack 100 may then be sealed in an outer package in block 230, for example, a foil pack or a plastic package, which is suitable for shipping and display. The sealed outer package inhibits the release of the volatiles prior to use. As an alternative or in addition, it is contemplated that a removable adhesive strip (not shown) may be placed over the micro-perforations and removed prior to use.

Although the above described stick pack 100 is formed with a single compartment for the composition 120, it is contemplated that the stick pack may be formed with multiple compartments. FIG. 18 illustrates an exemplary multi-compartment stick pack 250. In this embodiment, four separate compartments 252 are defined in the stick pack 250, each separate compartment delineated by sealed ends 255. Although four compartments are shown, more or fewer compartments are also clearly contemplated. The individual compartments may all be of similar or identical physical characteristics, e.g., micro-perforation 253 size, pattern, and depth. For example, separate adhesive strips (not shown) may be applied over the micro-perforations 253 in each compartment 252, such that the compartments 252 may be individually opened for releasing volatiles. This gives a user the option to open multiple compartments 252 initially to increase the rate of release of the composition, or to open each compartment 252 only after the previous compartment composition has been exhausted or lost its effectiveness.

Alternatively, the compartments 252 may be configured differently, for example, to accommodate different compositions 120. The multi-compartment stick pack 250 may therefore be readily designed to accommodate different compositions, with the micro-perforations in each compartment 252 tailored to produce a desired rate of release of the volatiles. As discussed above, a punched hole may be included for hanging or otherwise attaching the stick pack 250 to a device.

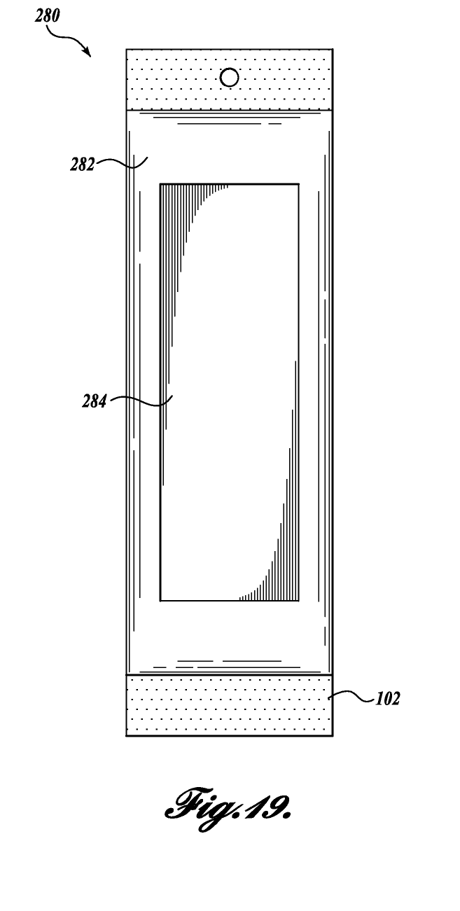

Another exemplary embodiment of a stick pack 280 is illustrated in FIG. 19. The stick pack 280 sachet is formed from a sheet material having at least an outermost lamina 282 as a barrier layer and an innermost lamina 284 as a sealing and releasing layer. This embodiment is similar to the stick pack 100 described above, except that rather than (or in addition to) a plurality of micro-perforations, windows are formed in the outermost lamina 282, defining an opening or “window” in the sachet that exposes the innermost lamina 284. The innermost lamina 284 may be permeable to the volatiles to permit a gradual release rate and/or may include micro-perforations (not shown) to further control the release rate. The innermost lamina 284 is therefore exposed for release of volatiles. A packaging or other external barrier (not shown) to prevent or mitigate release of the composition before deployment of the stick pack 280 could be provided. The packaging and stick pack are configured to maintain the integrity of the composition contents over time, e.g., during shipment and storage, such that the product will produce the desired release rate and retain its efficacy when the stick pack is deployed.

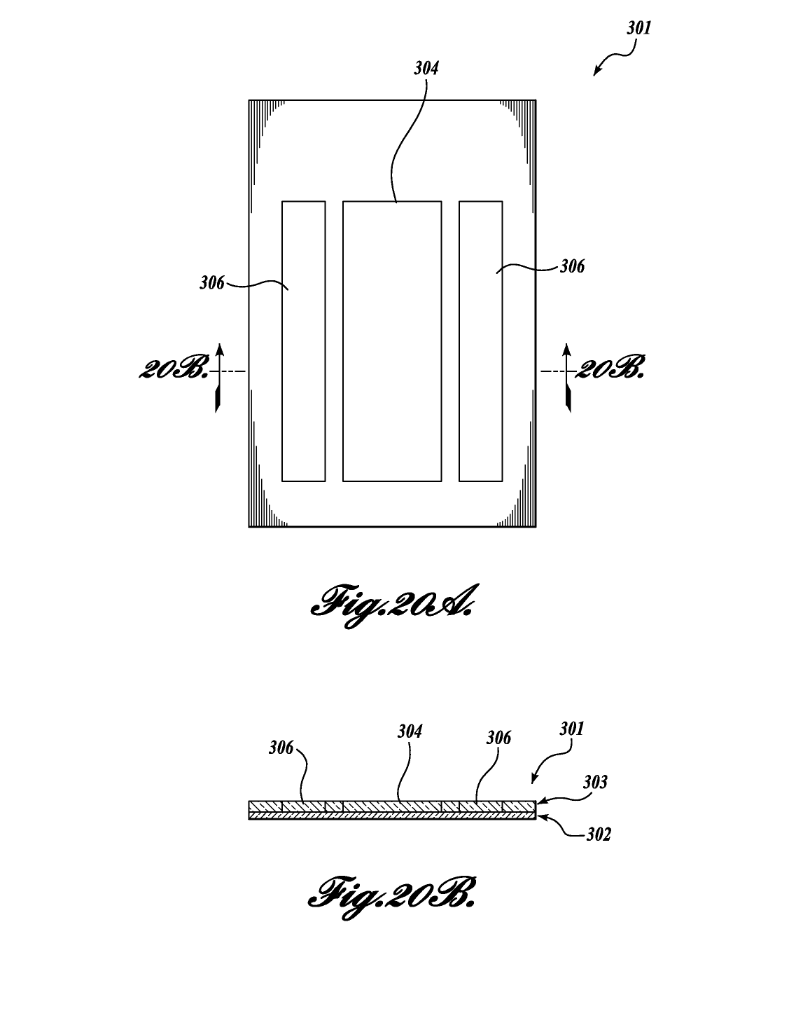

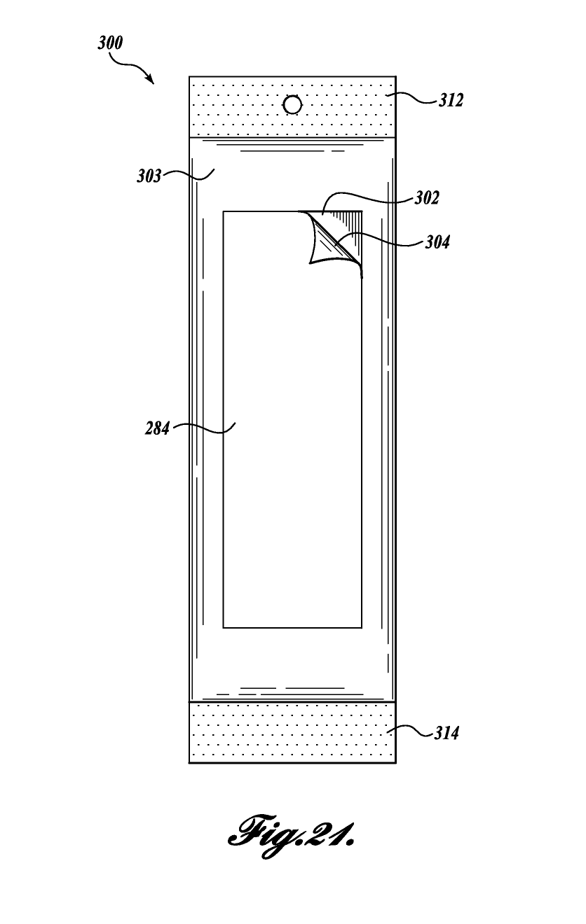

Another exemplary embodiment of a stick pack 300 is illustrated in FIGS. 20A, 20B, and 20C. FIG. 20A is a plan view of a portion of a sheet of material 301 for producing a single stick pack 300. It will be appreciated that the sheet of material would typically be configured on a continuous roll (not shown), and may include templates or room for multiple stick packs 300 across the width of the roll. FIG. 20B is a cross-sectional view of the unit template shown in FIG. 20A, with the depth dimension exaggerated for clarity.

In this embodiment, the inner layer 302 shown on the bottom in FIG. 20B is configured to define the inner lamina of the stick pack 300, and is adhered to an outermost layer 303. The outermost layer 303 includes one or more peel-away portions 304, 306 that are configured to be removed just prior to use, to open “windows” exposing a portion of the inner layer 302. The inner layer 302 may comprises a plurality of laminae, perhaps including micro-perforations as shown in FIG. 15, or may be a single layer without micro-perforations, and having a permeability to provide the desired release rate.

It is contemplated that the peel-away portions 304, 306 may be produced using different methods, as are known in the art. In an exemplary method the peel-away portions 304, 306 are created or defined by leaving a selected window portion of the inner layer un-laminated during the sheet-making process, and laser scoring or cutting the outer layer 303, without cutting the inner layer 302. The peel-away operation may be carried out as part of the film-making process, i.e., before the stick pack is formed, or may be left for the end-user to perform, for example, immediately before use.

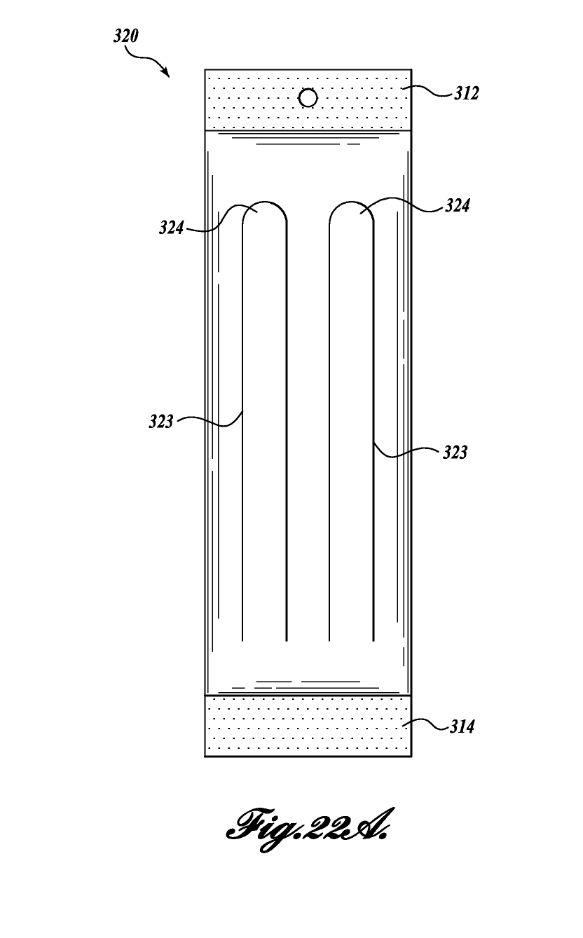

FIG. 21 shows a front view of the stick pack 300. End seals 312, 314 close the stick pack 300 at the top and bottom ends, and a longitudinal seal 314 closes the lateral edges to define the tube structure. The first peel-away portion 304 is shown partially removed to expose a portion of the inner layer 302.

This packaging arrangement provides the end-user with great control and flexibility in controlling the release rate of the composition contained therein, by allowing the end-user to determine how much of the peel-away portion 304 to peel down, and similarly how much of the back side peel-away portions 306 to peel down. For simplicity in manufacturing, in a current embodiment the peel-away portions are formed only on the front side of the stick pack.

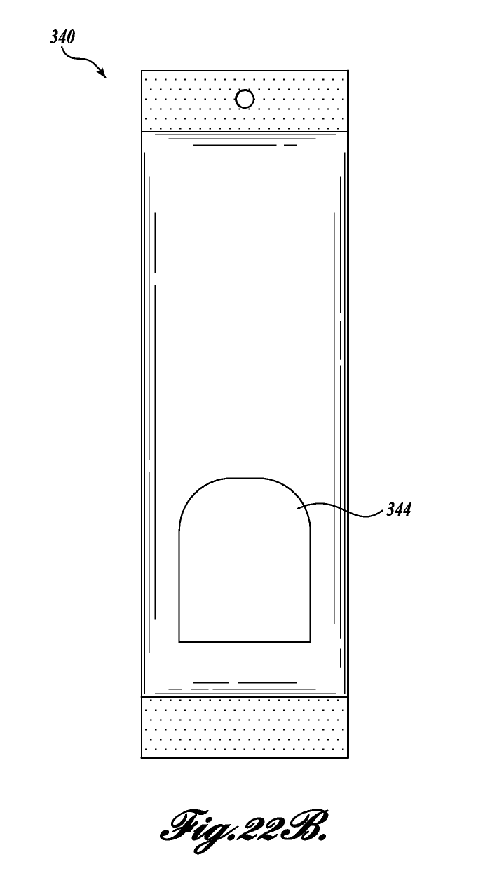

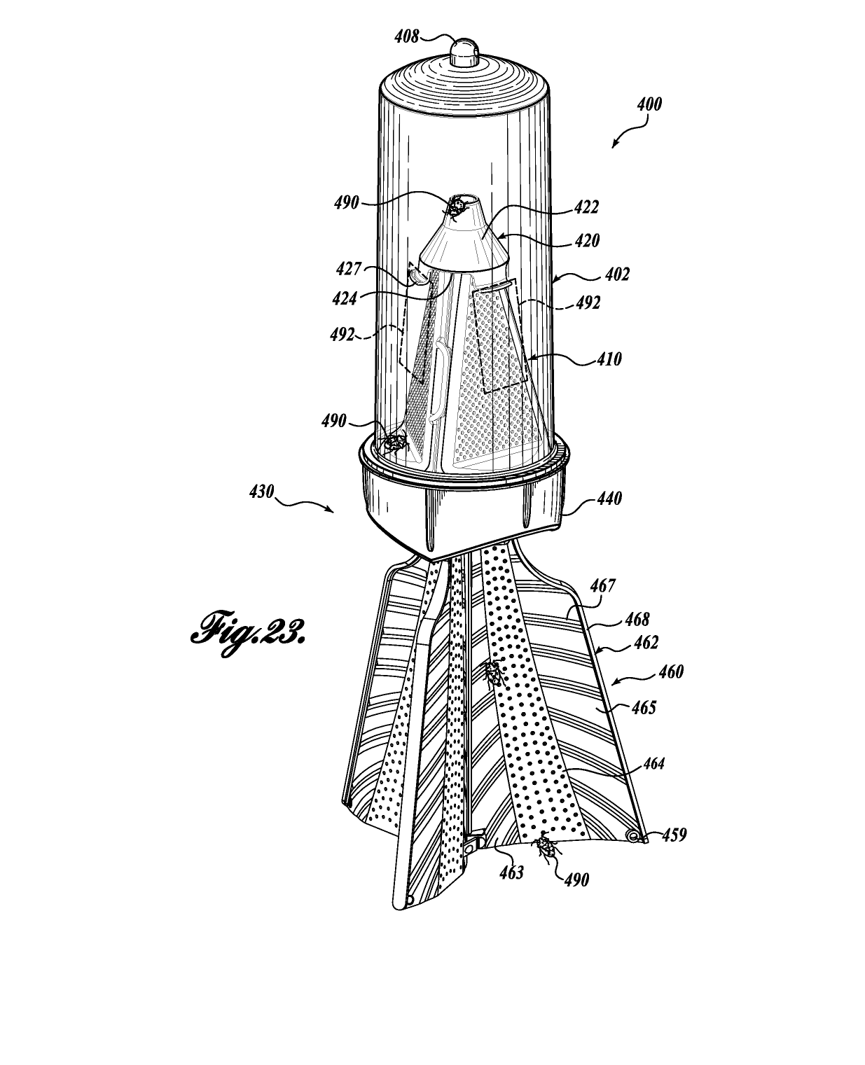

FIGS. 22A and 22B illustrate other embodiments of stick packs 320 and 340, respectively. The stick pack 320 includes two peel-away portions 324 defined by cuts or score lines 323. The score lines 323 are not closed, and therefore the peel-away portions 324 will generally remain attached to the stick pack 320. The multiple peel-away portions 324 allow an end-user to control the rate of release of volatiles from the stick pack 320 by peeling one or both of the peel-away portions 324 and/or by electing how far to pull the peel-away portion(s) 324 down the stick pack 320. Of course, more than two peel-away portions may used. In the embodiment in FIG. 22B, the stick pack 340 has a peel-away portion 344 that is much shorter than the length of the stick pack 340. Thus, for example, the user may more precisely control the location that volatiles are released from the stick pack 340.

The stick packs may alternatively be used to release a very large amount of insect semiochemical in the field, for example to repel target insects.

The stick packs provide a mechanism for very precisely controlling the release rate of semiochemicals contained in the stick pack. In exemplary uses, the stick packs may be used in insect traps to lure the target insect into the trap with an attractant.

Alternatively, stick packs containing repellant semiochemicals may be distributed about a particular perimeter to drive a target species away from a region, and/or to discourage the target insect from entering the region. For example, the stick packs may be placed around the points of entry into a building, or around a tent or other portable shelter.

The inclusion of antioxidants and/or insect-inactive oils with semiochemicals, such as essential oils and their compounds, may be the preferred method of dispensing some semiochemicals that are prone to degradation by oxygen, and ultraviolet light.

A non-limiting example of a stink bug trap that may be used with stink bug attractants is shown in FIG. 23. FIG. 23 is a perspective view of an insect trap 400 that is particularly suited for capturing insects such as stink bugs 490, for example, brown marmorated stink bugs and the like.

The trap 400 comprises an upper entrapment chamber 402, which in this embodiment is a generally cylindrical member that is open at a bottom end and closed at a top end. However, it will be readily apparent that the entrapment chamber may be alternatively shaped. The top end may optionally include a nib 408 to facilitate securing the trap 400 at a desired location. The entrapment chamber 402 may be formed from a transparent or translucent material to permit light to enter the entrapment chamber. It is also contemplated that the color of the entrapment chamber 402 will preferably be selected to attract the target species.

An inner member, herein referred to as the cone 410, is disposed in the entrapment chamber 402, tapering from a large opening at the bottom end disposed near the entrapment chamber bottom end, to a small opening at the top end disposed inside the entrapment chamber 402. A collar 420 is attached over a top end of the cone 410. The collar 420 in this embodiment includes a frustoconical upper portion 422 with a top edge that engages the cone 410 and a lower edge 424 that extends away from the cone 410. One or more hook members 427 extend down from the lower edge 424. Preferably, the surface of the frustoconical upper portion 422 is provided with a slippery or non-stick surface, to facilitate the target insects 490 falling near the bottom of the entrapment chamber 402, and to prevent insects 490 from crawling back out of the trap 400. For example, the upper portion 422 may include a layer of polytetrafluoroethylene powder (e.g., with a particle size of 0.1 to 3.0 microns) such as that marketed under the trade names Teflon® or Fluon®.

A lower base portion 430 of the trap 400 includes an annular lid 440 that is releasably attached to the entrapment chamber 402, and a vane assembly 460 assembled from a plurality of panels or vanes 462 (three shown) that extend downwardly from the annular lid 440.

As illustrated in phantom in FIG. 23, one or more sources of attractant 492 are preferably enclosed within the entrapment chamber 402. The attractant(s) disposed in the trap is typically packaged, formulated, or otherwise adapted to release gradually over time. Thus, in an exemplary embodiment, the attractant 492 is containerized in a porous package or in a package that the user opens at the top, although other controlled release disposers or devices may alternatively be used. In the illustrated embodiment, each attractant package 492 is retained on one of the hook members 427 of the collar 420. The attractant may be murgantiol or a murgantiol-containing synergistic composition, or other attractant as described herein. More generally, the attractant(s) may, for example, be disposed within the entrapment chamber 402, such as in a controlled release dispenser or device, between the cone 410 and the wall of the cylinder.

In the present embodiment the vanes 462 are designed with particular features that also take advantage of the stink bug's 490 behavioral tendencies. Stink bugs 490, for example, tend to alight on a surface, such as the ground, and to climb. For example, they may approach and even strike a vane 462 causing them to land at the base of the vane 462. The vanes 462 are designed to encourage the insect 490 to climb the vane 462 and to enter the entrapment chamber 402.

In this exemplary embodiment the vanes 462 are each curved in horizontal cross section to provide a more natural and organic shape that will be more inviting to the insect 490. For example, the vanes 462 may each be curved about a vertical axis. As seen most clearly in FIG. 23, the vanes 462 may include a center portion 464 that is substantially planar, an inner portion 463 extending inwardly from the center portion 464, and an outer portion 465 extending outwardly from the center portion 463, wherein the inner and outer portions 463, 465 are curved in horizontal cross section.

The vanes 462 are further provided with surface features that encourage and facilitate climbing. For example, the center portions 464 are provided with a plurality of apertures that extend along the length of the vane 462. The apertures facilitate climbing by providing a perch for the insects 490, and also permit air and light to penetrate, again providing a more organic-mimicking environment to encourage continued climbing. The inner portion 463 and outer portion 465 of each vane 462 further include a plurality of surface ridges 467 that extend generally from the center portion 464 to the inner and outer edges of the vane 462. The ridges 467 generally mimic a leaf vein structure and further facilitate climbing the vane 462, and gently encourage the insects 490 toward the center portion 464. The outer edge of each vane 462 is further provided with a flange 468, such that climbing insects 490 are directed upwardly.

Optionally, the vanes 462 may further comprise means for fixing the trap 400 at a particular location. For example, apertures 459 in the lower outside corners of each vane 462 in the current embodiment may be provided with a string, cable, tie wrap, or the like (not shown) that can be secured to a fixed object, such as a portion of a tree, a pipe, etc.

It is to be understood that the trap of FIG. 23 is only exemplary and not limiting.

The use of the term “or” in the claims is used to mean “and/or” unless explicitly indicated to refer to alternatives only, or the alternatives are mutually exclusive, although the disclosure supports a definition that refers to only alternatives and “or.”

Throughout this application, the term “about” is used to indicate that a value includes the standard deviation of error for the device or method being employed to determine the value. In any embodiment discussed in the context of a numerical value used in conjunction with the term “about,” it is specifically contemplated that the term about can be omitted.

Following long-standing patent law, the words “a” and “an,” when used in conjunction with the word “comprising” in the claims or specification, denotes one or more, unless specifically noted.

### EXAMPLES

### Materials and Methods

Headspace Sampling of Essential Oils and Collection of Insects

Headspace volatiles from several essential oils (1 ml each in a closed 20 ml glass vial) were sampled via Solid-Phase Micro-Extraction (SPME) (CAR/PDMS, 85 μm, Supelco, Bellefonte, Pa.) for 30 sec before GC-EAD and GC-MS analyses.

BMSB nymphs and adults for electrophysiological study were taken from a lab colony originally collected from Pennsylvania, U.S.A., during the overwintering period.

GC-EAD/MS Analyses of Essential Oils

SPME samples of the various essential oils were injected splitless into a Varian CP-3800 GC equipped with a polar column (HP-INNOWAX; 30 m×0.53 mm×1.0 μm film thickness; Agilent Technologies, Wilmington, Del., USA) and a 1:1 effluent splitter that allowed simultaneous flame ionization detection (FID) and electroantennographic detection (EAD) of BMSB adult or nymph antenna to the separated volatile compounds. Helium was used as the carrier gas, and the injector and detector temperatures were 250° C. and 300° C., respectively. Column temperature was 50° C. for 1 min, rising to 240° C. at 10° C./min, and then held for 10 min. The outlet for the EAD was held in a humidified air stream flowing at 0.5 m/sec over the antennal preparation. EAD recordings were made using silver wire-glass capillary electrodes filled with Beadle-Ephrussi Ringer on freshly cut antennae. The antennal signals were stored and analyzed on a PC equipped with a serial IDAC interface box and the program EAD ver. 2.5 (Syntech, Hilversum, The Netherlands). The same GC-EAD recording procedure was also employed on the BMSB antennae against a synthetic mixture of the Spined soldier bug aggregation pheromone components (trans-2-hexenal, benzyl alcohol, and α-terpineol; 200 ng/μl each in hexane, 2 μl injected).

Antennally active compounds (FID peaks) in the SPME samples of essential oils were identified by GC-MS on an HP 6890 GC series coupled with an HP 5973 Mass Selective Detector using the same type of GC column and conditions as described above. Compounds were identified by comparison of retention times with those of authentic standards and with mass spectra of standards.

Chemical Standards and Essential Oils

The following authentic chemical standards for chemical identification or field trapping experiments were obtained from various commercial and noncommercial sources: benzyl acetate (99%), l-carvone (98.5%), eugenol (98%), geraniol (98%), methyl benzoate (99%), trans-2-hexenal (98%), benzyl alcohol (99%), α-terpineol (96%), methyl salicylate (99%), and β-caryophyllene (98%) were obtained from Sigma-Aldrich Chemical (Milwaukee, Wis.); E/Z-citral (>90%), menthone (>96%), and pulegone (>96%) from Vigon International, Inc., East Stroudsburg, Pa.

The following essential oils tested were purchased from Lorann Oil, Inc. (Lansing, Mich.): clove oil, geranium oil, lemongrass oil, pennyroyal oil, rosemary oil, spearmint oil, wintergreen oil, and ylang ylang oil.

Field Trapping Experiments

Two field trapping experiments were carried out from mid-July to late September 2011 on a farm surrounded with deciduous trees and secured with a fence line in Prince George's County, Maryland, USA. In order to test potential repellency of essential oils or some strongly EAD-active synthetic candidate compounds, the commercially available Rescue® stink bug traps (patent Nos.: U.S. D645,534S and U.S. D645,535S) baited with the known stink bug attractants (methyl 2E,4E,6Z-decatrienoate and methyl 2E,4Z-decadienoate each was released from a separated PE-porch) were used. Traps were hung 1.5-2.0 m above the ground on either tree branches or fences ca. 8-10 m apart between each trap and at least 20 m between sets. For each trapping experiment, six sets of traps (i.e., six physical replicates of each treatment) were deployed with their initial trap positions within each set being randomized. To minimize positional effects and obtain more replications, stink bug collections and trap re-randomization were carried out when ≧10 stink bugs were caught in the best traps. Each replicate lasted several days to a week depending on stink bug activity. Captured stink bugs were removed from the traps and kept in the zip-bags before returning to the laboratory for recording of the species, gender and development status (nymphs, adult males/females), and catch. Repellent candidates (individuals or mixtures) were released from 4 mil polyethylene bags (2.3×5.5 cm); they were employed inside the same trap as the attractant lures.

Experiment 1 tested eight individual essential oils [pennyroyal (55 mg/d), lemongrass (25 mg/d), spearmint (80 mg/d), clove (14 mg/d), wintergreen (63 mg/d), rosemary (105 mg/d), ylang ylang (37 mg/d), and geranium (15 mg/d)] and a synthetic mixture of the Spined soldier bug aggregation pheromone (i.e. E2-hexenal, α-terpineol and benzyl alcohol at 1:1:0.1 ratio; 35 mg/d), plus a blank control against the Rescue® 7-week stink bug attractant lures using the Rescue® stink bug traps from Jul. 14 to Aug. 15, 2011.

Experiment 2 tested nine selected EAD-active synthetic compounds identified from repellent essential oils: l-carvone (45 mg/d), eugenol (11 mg/d), E/Z-citral (22 mg/d), pulegone (85 mg/d), P/1-menthones (95 mg/d), methyl benzoate (135 mg/d), geraniol (12 mg/d), β-caryophyllene (55 mg/d), and methyl salicylate (95 mg/d); a mixture of three essential oils (30 mg/d; lemongrass, spearmint and clove at 1:1:1), plus a blank control against the Rescue® 7-week stink bug attractant lures using the Rescue® stink bug traps in September 2011.

Statistical Analysis

Trap catch data (number of stink bugs/trap/visit) were transformed by log(X+1) and the transformed means were analyzed by ANOVA, followed by the Duncan's multiple-range test (SPSS 16.0 for Windows) at α=0.05.

Results

Field Bioassays of the Essential Oils

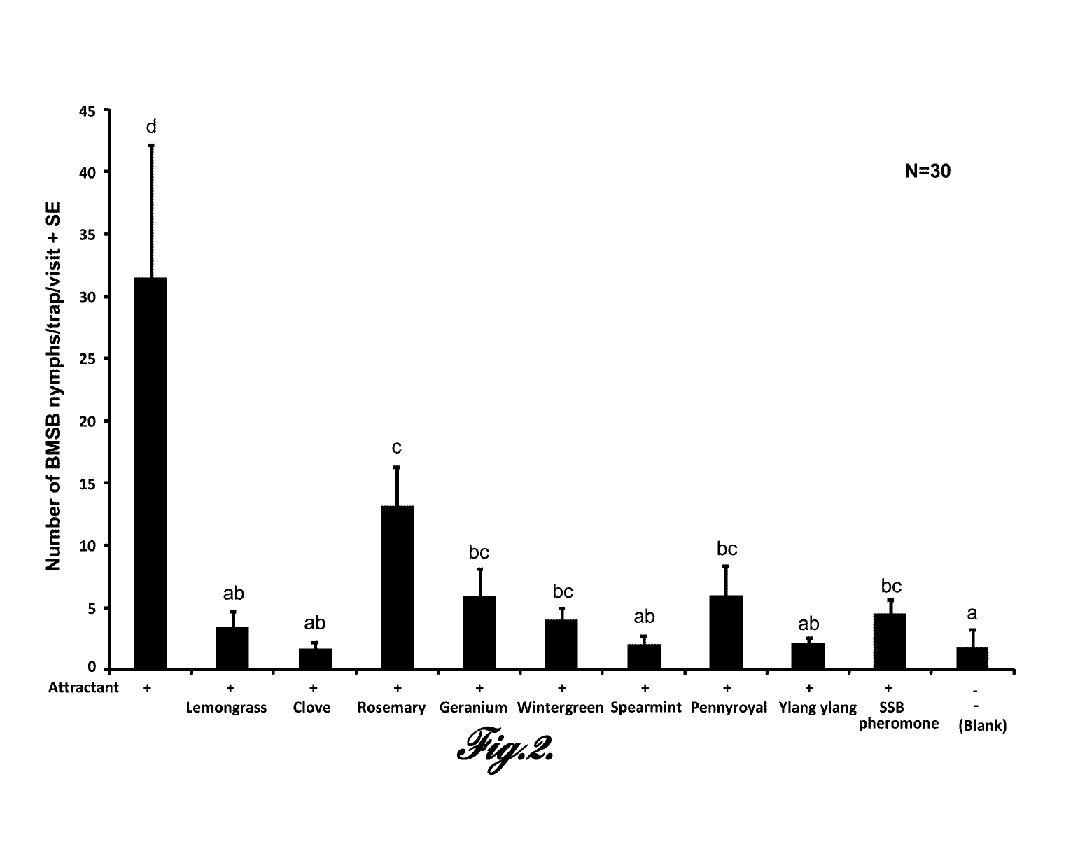

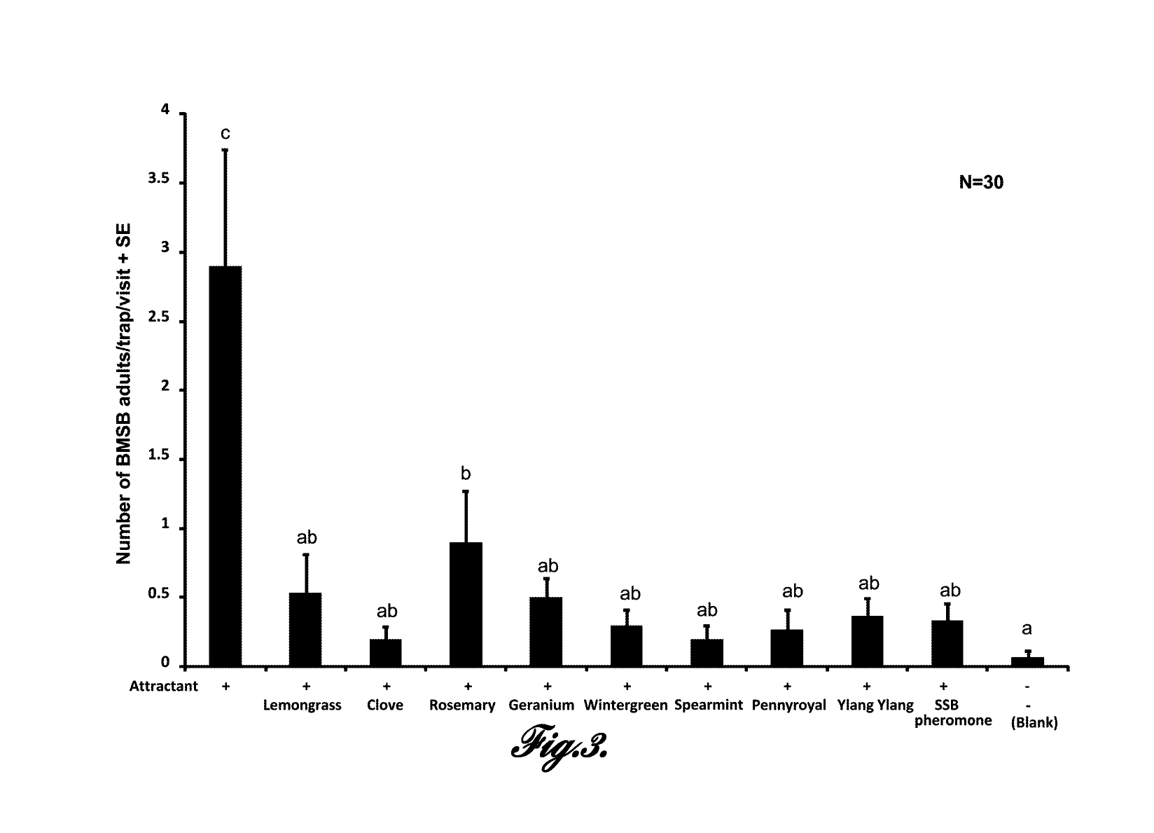

Eight essential oil individuals and one essential oil mixture were tested in Experiments 1-2 against the Rescue® 7-week stink bug attractant lures from Mid-July to late September, 2011. All the tested essential oil individuals and the mixture showed significant repellency on both BMSB nymphs (FIG. 2) and adults (FIG. 3) to the stink bug attractants. Clove oil, lemongrass oil, ylang ylang oil and spearmint oil at 14-80 mg/day release rates almost blocked the attraction of these stink bugs (both nymphs and adults) to their stink bug attractants (FIGS. 2-3). Wintergreen oil, geranium oil, pennyroyal oil and rosemary oil also resulted in 60-85% trap catch reductions. Surprisingly, a synthetic mixture of the Spined soldier bug aggregation pheromone also showed significant repellency on the BMSB nymphs and adults (FIGS. 2-3). Unlike the BMSB stink bug, which is a serious pest, the Spined soldier bug (though a species of the stink bug family) is a common beneficial insect predator that can attack many pest insect larvae and eggs, including those of the BMSB.

GC-EAD/MS Analyses of Behaviorally Active Essential Oils

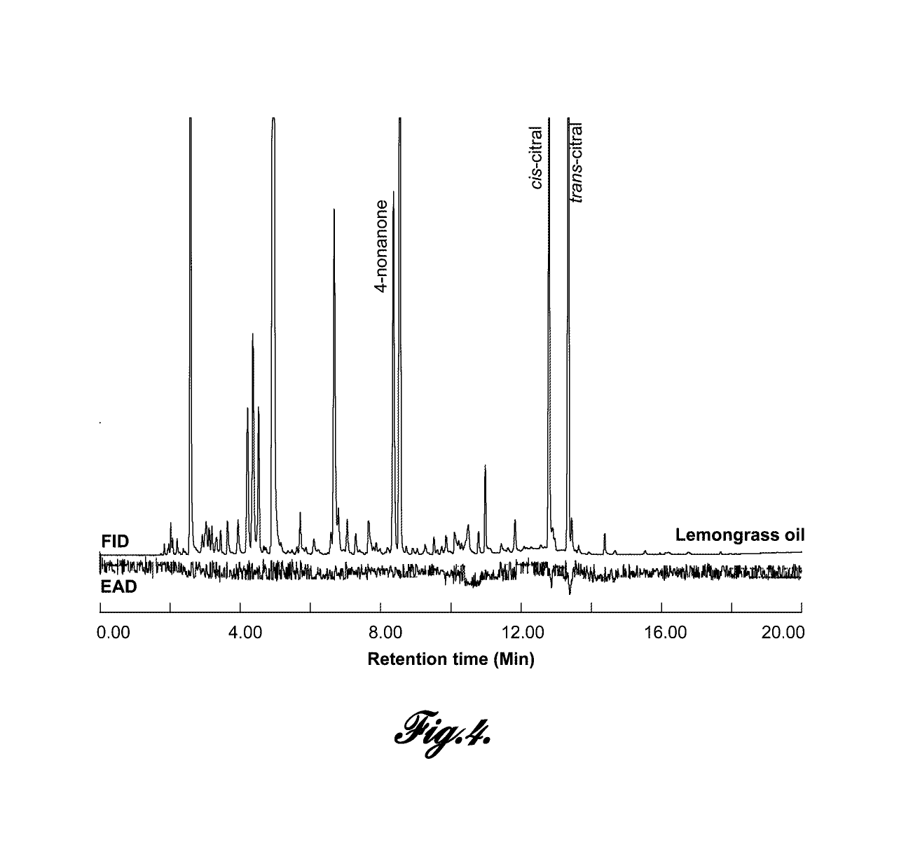

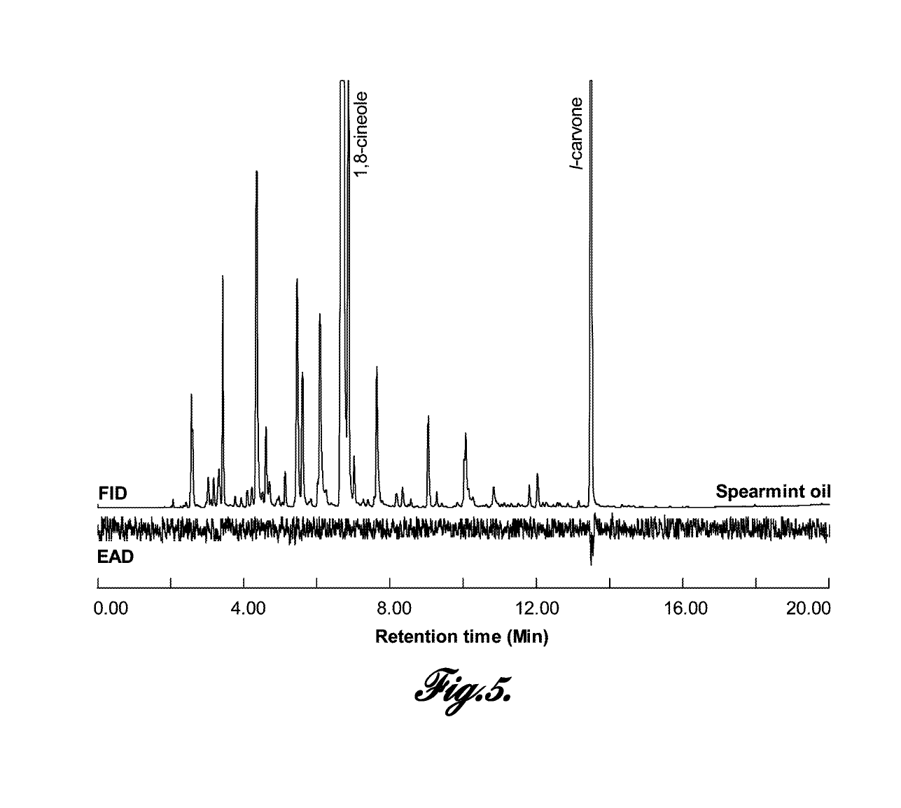

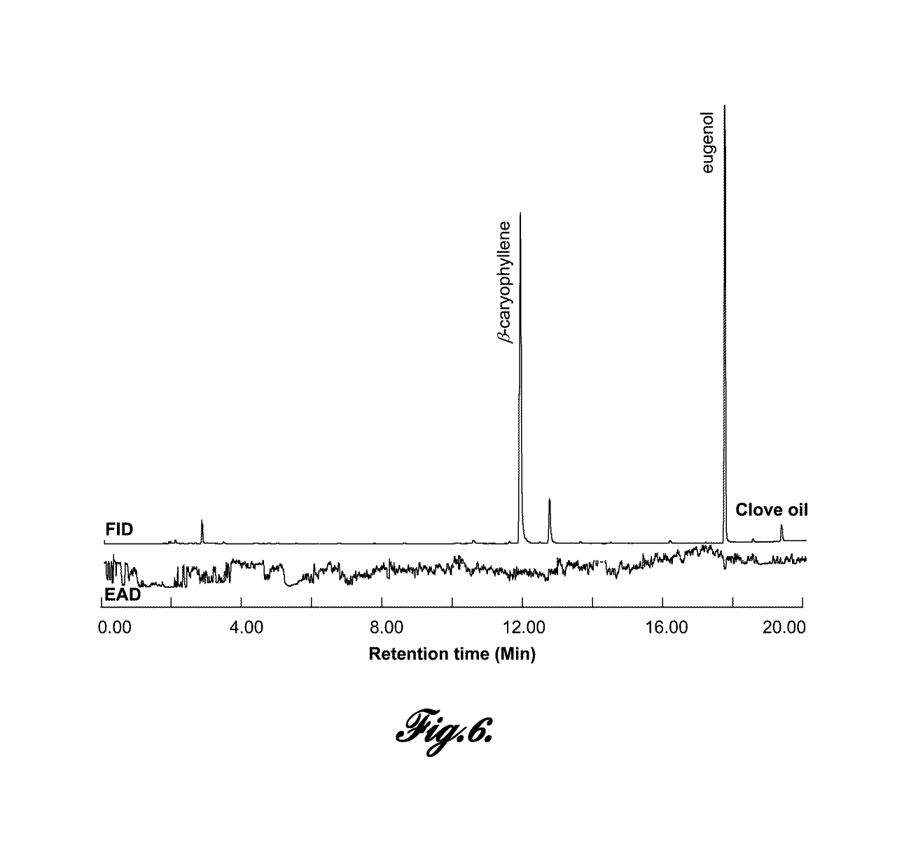

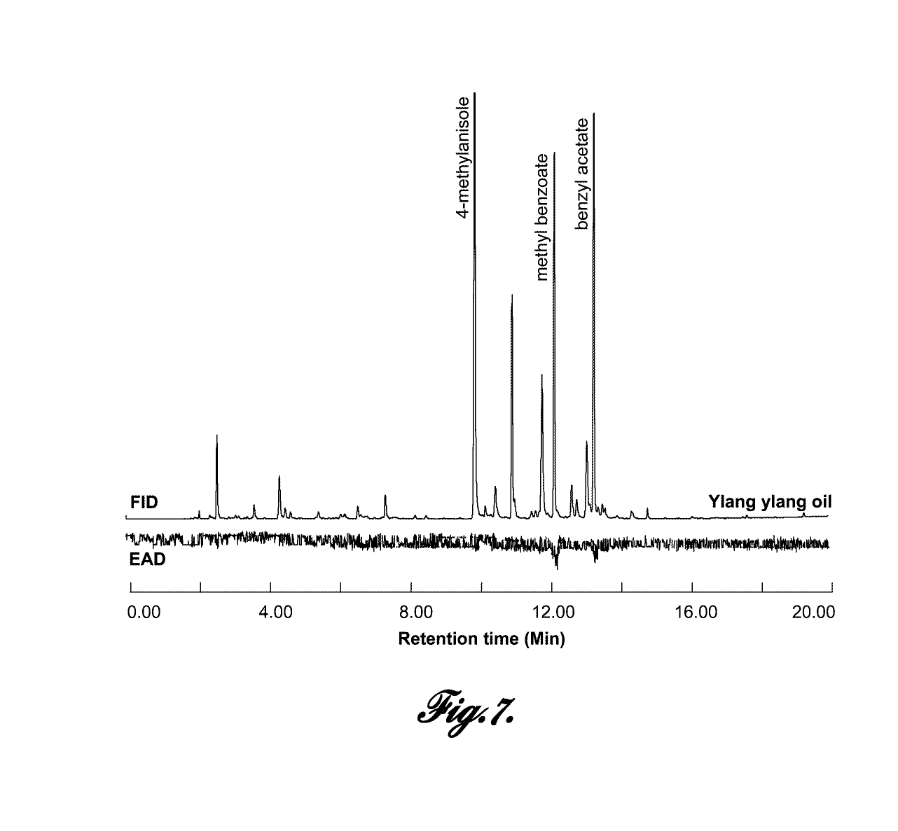

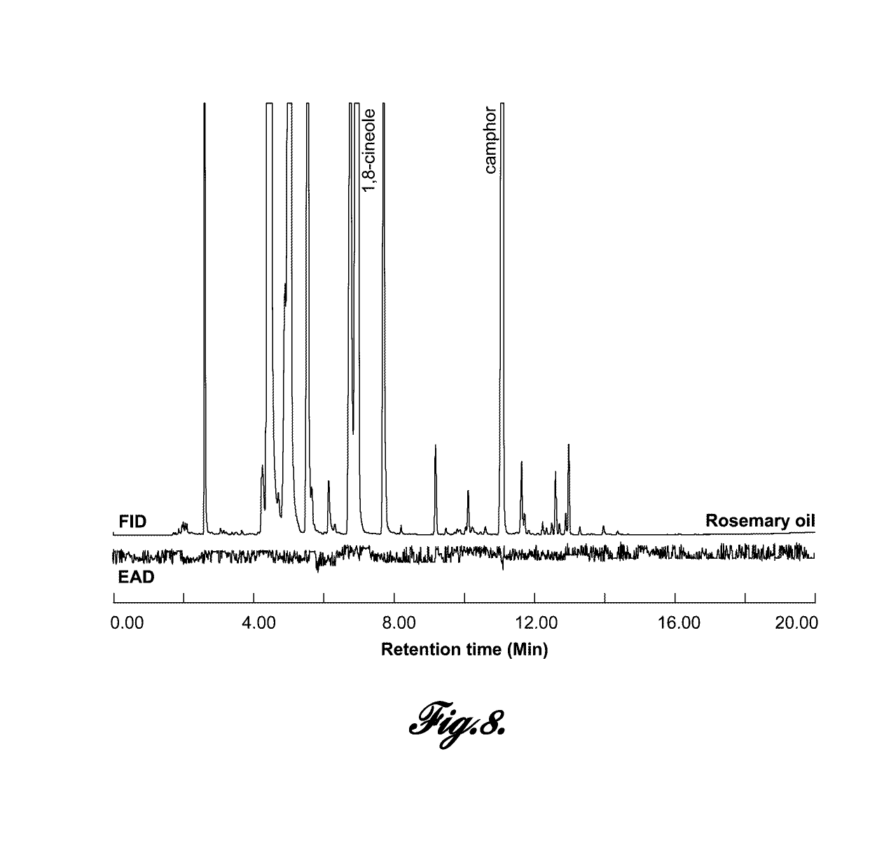

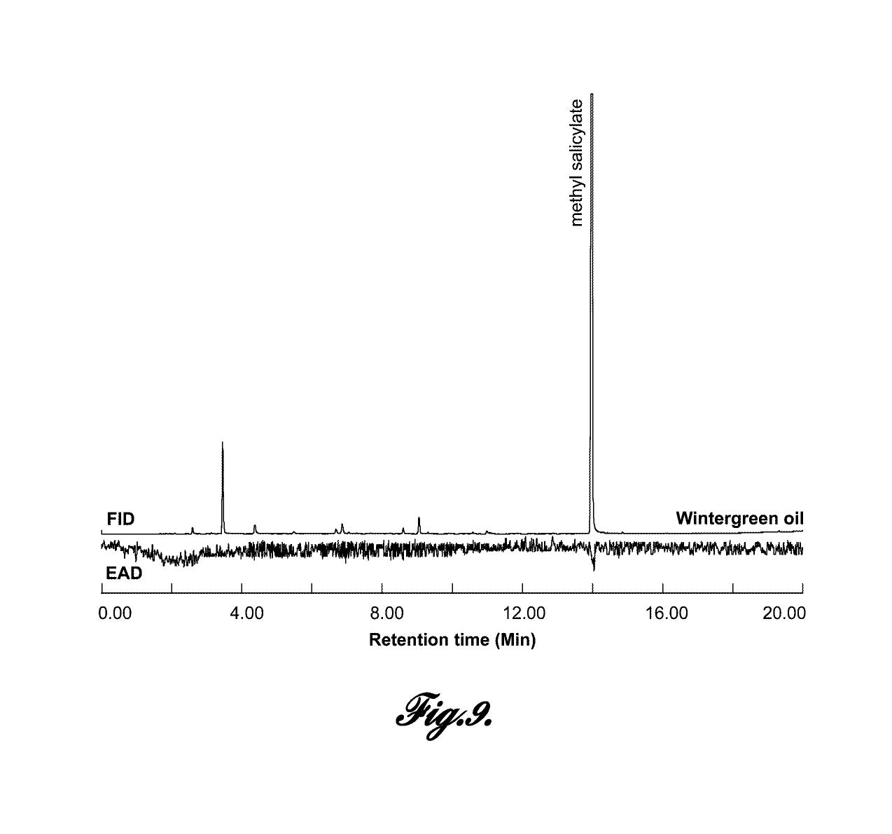

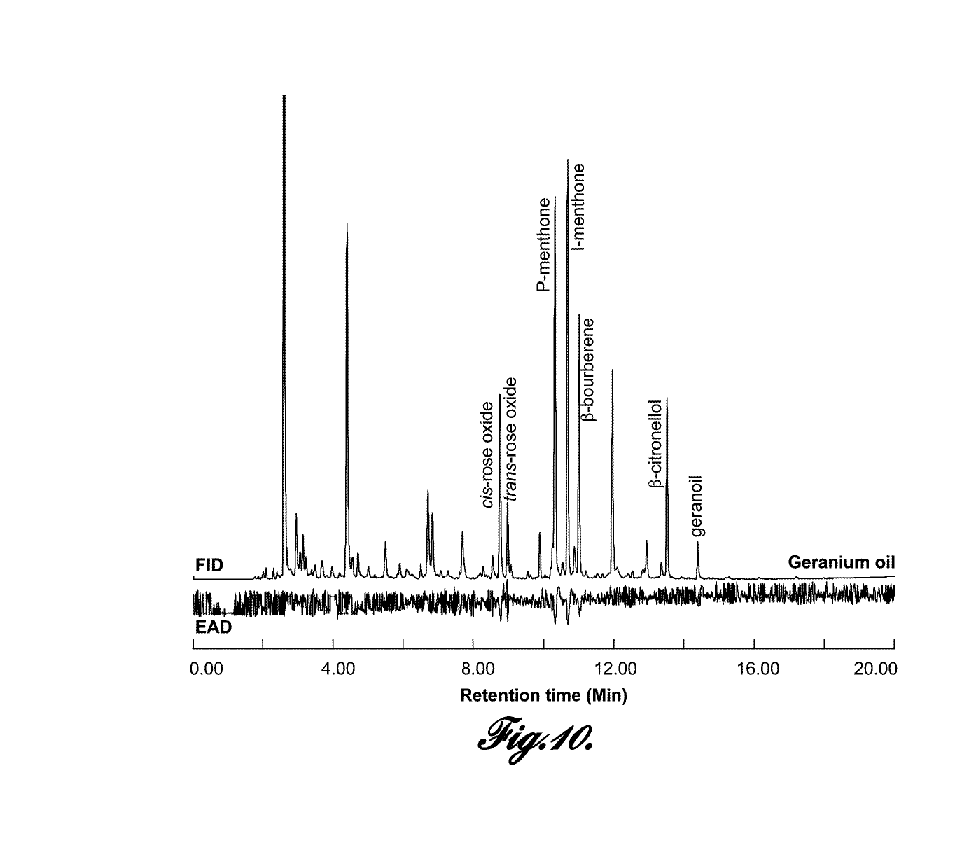

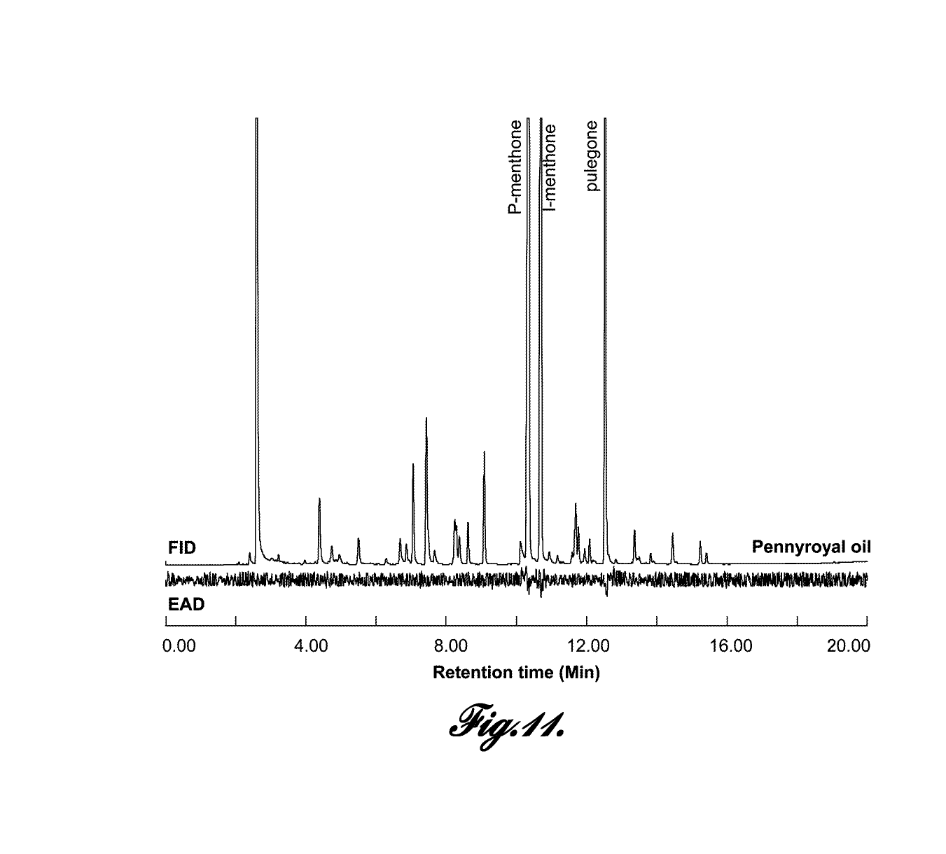

In order to identify the potential chemical compositions from the strong behaviorally repellent essential oils that might be responsible for stink bug repellency, a series of GC-EAD/MS analyses were carried out on eight selected essential oil headspace samples against BMSB (nymphs and adults) antennae. As shown in FIG. 4, antennae of BMSB strongly responded to two major components, E-citral (trans-citral) and Z-citral (cis-citral), and weakly to a minor component, 4-nonanone from the lemongrass oil. One major component, l-carvone, from spearmint oil (FIG. 5); one of the two major components, eugenol, from clove oil (FIG. 6); three major components, 4-methylanisole, methyl benzoate, and benzyl acetate from ylang ylang oil (FIG. 7); two major compounds, 1,8-cineole and camphor from rosemary oil (FIG. 8); the dominant component, methyl salicylate, from wintergreen oil (FIG. 9); seven compounds from geranium oil (cis/trans-rose oxides, P/I-menthones, β-bourberene, β-citronellol, and geraniol) (FIG. 10); and three major components, P/I-menthones and pulegone, from pennyroyal oil (FIG. 11), elicited significant (strongly or weakly) EAD-responses by BMBS adult and nymph antennae. These antennally-active compounds were identified using GC-MS by comparison of retention times with those of authentic standards and with the mass spectra of standards. GC-EAD on a synthetic mixture of the Spined soldier bug aggregation pheromone components showed strong antennal responses to trans-2-hexenal and benzyl alcohol, and a weak activity to α-terpineol by BMSBs antennae (FIG. 12); indicating that BMSB antennae do have olfactory receptor neurons to detect the aggregation pheromone components of its potential predators.

In short, around 20 EAD-active compounds were identified from eight selected behaviorally strong active repellent essential oils for BMSBs (see Table 1 for summary). No difference in EAD responses between nymphs and adults; and between the sexes was detected.

TABLE 1
Stink bug EAD-active compounds from the behaviorally
repellent essential oils or synthetic SSB pheromone

| | Lemon | Spear- | Penny- | Ylang | Gera- | Rose- | | Winter- | SSB |
|---|---|---|---|---|---|---|---|---|---|
| Chemical | grass | mint | royal | ylang | nium | mary | Clove | green | pheromone |
| 1,8-cineole | | * | | | | * | | | |
| 4-nonanone | * | | | | | | | | |
| cis-rose oxide | | | | | *** | | | | |
| trans-rose oxide | | | | | * | | | | |
| 4-methylanisole | | | | * | | | | | |
| P-menthone | | | *** | | *** | | | | |
| I-menthone | | | *** | | *** | | | | |
| camphor | | | | | | *** | | | |
| β-caryophyllene | | | | | | | * | | |
| β-bourbernene | | | | | *** | | | | |
| methyl benzoate | | | | *** | | | | | |
| benzyl acetate | | | | *** | | | | | |
| β-citronellol | | | | | * | | | | |
| methyl salicylate | | | | | | | | *** | |
| l-carvone | | *** | | | | | | | |
| pulegone | | | *** | | | | | | |
| Z-citral | *** | | | | | | | | |
| E-citral | *** | | | | | | | | |
| geraniol | | | | | * | | | | |
| eugenol | | | | | | | *** | | |
| trans-2-hexenal | | | | | | | | | *** |
| benzyl alcohol | | | | | | | | | *** |
| α-terpineol | | | | | | | | | * |

* weakly (or occasionally) active;
*** strongly (and repeatedly) active.
Chemicals in bold were tested in the field.
SSB pheromone: Spined soldier bug aggregation pheromone

Field Bioassays of the EAD-Active Synthetic Compounds

Nine synthetic EAD-active compounds (identified from the repellent essential oils) were tested in a field-trapping experiment (experiment 2) against the Rescue® 7-week stink bug attractants. Traps baited with the stink bug attractants plus these synthetic repellent candidates seemed to catch significantly less BMSB than did the attractants alone.

### DISCUSSION

This patent application discloses various essential oils and their chemical compositions are repellent candidates (or attraction-inhibitors) for BMSB (both nymphs and adults). All eight essential oils tested showed significant repellency on BMSBs to their powerful attractants. Of these, clove oil (Myrtaceae), lemongrass oil (Poaceae), ylang ylang oil (Annonaceae), and spearmint oil (Lamiaceae) almost blocked the attraction of BMSBs to their stink bug attractants (M246DT/M24DD). The remaining essential oils showed 50-85% repellency effect. An essential oil mixture [clove, spearmint and lemongrass at 1:1:1) showed remarkably strong repellency; in fact, it totally blocked the attraction of BMSBs to their stink bug attractants (M246DT/M24DD); and strongly interrupted the attraction of BMSBs to their powerful attractants. Such significant repellency against the strong stink bug attractants clearly indicated that these essential oils (individuals or in mixtures) would have a great potential to repel these pestiferous BMSBs from their food sources and overwintering sites. One of the advantages for essential oil mixtures is the potential synergistic (or at least additive) effect among the individual oils and release rate complementary effect of more or less volatile compounds from different oils for a long lasting repellency.

GC-EAD analysis clearly showed that the BMSB antennae do have olfactory receptor neurons for detecting various essential oils and some of their volatile compounds (FIGS. 4-11; and Table 1). Nine EAD-active compounds were identified from eight selected behaviorally strong repellent essential oils by BMSBs (Table 1). No difference in EAD responses among BMSB nymphs and male/female adults was detected, suggesting that both stages or sexes share many common receptor neuron types and respond to the same or similar volatile repellent compositions. EAD activity is an indicator that the compound may be behaviorally active; and in cases of repellent essential oils, EAD-active compounds are likely responsible for the oil repellency to the stink bugs. In fact, many of the nine synthetic EAD-active compounds tested showed a significant repellency on BMSBs in a preliminary field trapping experiment. These EAD-active and behaviorally repellent volatile compounds showed both similarity and diversity in terms of chemical structures, including monoterpene aldehydes, alcohols, and ketones and phenolproponoids among many others (Table 1).

Interestingly, a synthetic mixture of the Spined soldier bug (SSB) aggregation pheromone also showed significant repellency on the BMSB nymphs and adults (FIGS. 2-3). GC-EAD analysis indicated that BMSB antennae do have olfactory receptor neurons to detect the aggregation pheromone components of this predacious bug (FIG. 12). The Spined soldier bug (though a species of the stink bug family) is a common beneficial insect predator that can attack many pest insect larvae and eggs, including those of the BMSB. It is beneficial for the BMSBs to be able to detect and avoid the aggregation pheromone of a potential predator. On the other hand, such repellency effect can be explored as an environmentally sound pest control strategy by attracting the naturally distributed predators (Spined soldier bugs) into the target area (such as vegetable garden or orchard) for feeding on the BMSBs already on the plants, and by repelling the BMSBs from entering the target area. Furthermore, these natural or synthetic repellents can also be deployed in cooperation with pheromone attractant-baited traps (set up around the target plants or trees to pull the stink bugs away from the targets) for these stink bugs in a push-pull tactic, which would likely enhance the pest control efficacy against these pestiferous stink bugs.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for repelling an insect, comprising:
releasing into a space a repellent composition comprising at least one of (a) or (b), or both (a) and (b):
(a) a first essential oil selected from the group consisting of lemongrass oil, ylang ylang oil, clove oil, geranium oil, rosemary oil, spearmint oil, wintergreen oil, and pennyroyal oil, or any combination thereof;
(b) a second essential oil comprising at least one compound selected from the group consisting of I-menthone, P-menthone, eugenol, E-citral, Z-citral, pulegone, methyl benzoate, l-carvone, methyl salicylate, benzyl acetate, β-caryophyllene, geraniol, camphor, cis-rose-oxide, an isomer thereof, or any combination thereof, wherein the compound is a major constituent of the second essential oil;
wherein the repellent composition is comprised in a controlled release device having at least one aperture configured to achieve a desired rate of release of the repellent composition in a volatilized state into the space; and
repelling from the space an insect belonging to the insect order Heteroptera, the Pentatomoidea superfamily, including stink bugs and shield bugs; the Superfamily Coreoidea, including squash bugs, or the insect family Miridae, including plant bugs, wherein each essential oil of (a) or (b) acts to repel the insect.

2. The method of claim 1, wherein the repellent composition comprises spearmint oil in an amount effective to repel the insect.

3. The method of claim 1, wherein the repellent composition comprises spearmint oil, clove oil, and lemongrass oil in an amount effective to repel the insect.

4. The method of claim 1, wherein the repellent composition comprises l-carvone in an amount effective to repel the insect.

5. The method of claim 1, wherein the repellent composition comprises two or more first essential oils of (a).

6. The method of claim 1, wherein the repellent composition comprises two or more second essential oils of (b).

7. The method of claim 1, wherein the repellent composition comprises at least one first essential oil of (a) and at least one second essential oil of (b).

8. The method of claim 1, wherein the repellent composition further comprises at least one compound selected from the group consisting of I-menthone, P-menthone, eugenol, E-citral, Z-citral, pulegone, methyl benzoate, l-carvone, methyl salicylate, benzyl acetate, β-caryophyllene, geraniol, camphor, cis-rose-oxide, an isomer thereof, or any combination thereof.

9. The method of claim 1, wherein the controlled release device comprises a polymeric sheet having a means for permitting the repellent composition in a volatilized state to pass therethrough.

10. The method of claim 9, wherein the polymeric sheet comprises a plurality of lamina.

11. The method of claim 10, wherein an innermost lamina of the plurality of lamina is semi-permeable such that the repellent composition in a volatilized state can pass through the innermost lamina.

12. The method of claim 9, wherein the means for permitting the repellent composition to pass therethrough comprises a plurality of micro-perforations.

13. The method of claim 12, wherein the polymeric sheet further comprises an innermost lamina and wherein at least some of the plurality of micro-perforations do not penetrate an innermost lamina of the polymeric sheet.

14. The method of claim 1, wherein the insect is selected from the group consisting of stink bugs, shield bugs, squash bugs, and plant bugs or any combination thereof.

15. The method of claim 1, wherein the insect is selected from a green stink bug (Acrosternum hilare (Say)); any of several species of Euschistus spp. including a brown stink bug (Euschistus servus (Say)), E. tristigmus, E. conspersus, E. variolarius, E. politus, or E. heros; a southern green stink bug (Nezara viridula (L.)); an eastern green stink bug (Nezara antennata); a spined soldier bug (Podisus maculiventris); an East Asian stink bug, a yellow-brown stink bug, a brown marmorated stink bug (Halyomorpha halys); a red-shouldered stink bug (Thyanta pallidovirens); a globular stink bug (Megacopta punctatissimum); a white-spotted stink bug (Eysarcoris ventralis); a fruit-piercing stink bug (Glaucias subpunctatus); a red-striped stink bug (Graphosoma rubrolineatum); a brown marmorated stink bug (Halyomorpha mista); a rice stink bug (Lagynotomus elongates, Oebalus pugnax); a two-spotted stink bug (Perillus bioculatus); a conchuela stink bug (Chlorochroa ligata); a Uhler's stink bug (Chlorochroa uhlerii); a Say's stink bug (Chlorochroa sayi); a brown-winged green stink bug (Plautia stali (Scott)); a boxelder bug (Boisea trivittata (Say)); a species of Banasa dimidiata (Say); a species of B. calva (Say); a species of B. euchlora Stål; a Harlequin bug (Murgantia histrionica); a Kudzu bug [Megacopta cribraria (Fabricius)]; a species of Anasa spp., including A. tristis (DeGeer); a species from the genus Leptoglossus; a species of Lygus spp., including a tarnished plant bug (L. lineolaris), a western tarnished plant bug (L. hesperus), a European tarnished plant bug (L. ruguilipnennis), a green leaf bug (L. lucorum); a species of apple dimpling bug (Campylomma spp.); a species of mosquito bug (Helopeltis spp.); a species of a honeylocust plant bug (Diaphnocoris spp.); a species of green mirid (Creontiades spp.); and a species of potato mirid (Calocoris spp).

16. A method for repelling insects, comprising:
releasing into a space a repellent composition comprising at least one compound selected from the group consisting of I-menthone, P-menthone, eugenol, E-citral, Z-citral, pulegone, methyl benzoate, l-carvone, methyl salicylate, benzyl acetate, β-caryophyllene, geraniol, camphor, cis-rose-oxide, an isomer thereof, or any combination thereof;
wherein the repellent composition is comprised in a controlled release device having at least one aperture configured to achieve a desired rate of release of the repellent composition in a volatilized state into the space; and
repelling from the space an insect belonging to the insect order Heteroptera, the Pentatomoidea superfamily, including stink bugs, and shield bugs; the Superfamily Coreoidea, including squash bugs, or the insect family Miridae, including plant bugs, wherein each synthetic compound acts to repel the insect.

17. The method of claim 16, wherein the repellent composition comprises l-carvone in an amount effective to repel the insect.

18. The method of claim 16, wherein the repellent composition further comprises at least one of (a) or (b), or both (a) and (b):
(a) a first essential oil selected from the group consisting of lemongrass oil, ylang ylang oil, clove oil, geranium oil, rosemary oil, spearmint oil, wintergreen oil, and pennyroyal oil, or any combination thereof;
(b) a second essential oil comprising at least one compound selected from the group consisting of I-menthone, P-menthone, eugenol, E-citral, Z-citral, pulegone, methyl benzoate, l-carvone, methyl salicylate, benzyl acetate, β-caryophyllene, geraniol, camphor, cis-rose-oxide, an isomer thereof, or any combination thereof, wherein the compound is a major constituent of the second essential oil.

19. The method of claim 18, wherein the repellent composition comprises spearmint oil in an amount effective to repel the insect.

20. The method of claim 18, wherein the repellent composition comprises spearmint oil, clove oil, and lemongrass oil in an amount effective to repel the insect.

21. The method of claim 18, wherein the repellent composition comprises two or more first essential oils of (a).

22. The method of claim 18, wherein the repellent composition comprises two or more second essential oils of (b).

23. The method of claim 18, wherein the repellent composition comprises at least one first essential oil of (a) and at least one second essential oil of (b).

24. The method of claim 16, wherein the controlled release device comprises a polymeric sheet having a means for permitting the repellent composition in a volatilized state to pass therethrough.

25. The method of claim 24, wherein the polymeric sheet comprises a plurality of lamina.

26. The method of claim 25, wherein an innermost lamina of the plurality of lamina is semi-permeable such that the repellent composition in a volatilized state can pass through the innermost lamina.

27. The method of claim 24, wherein the means for permitting the repellent composition to pass therethrough comprises a plurality of micro-perforations.

28. The method of claim 27, wherein the polymeric sheet further comprises an innermost lamina and wherein at least some of the plurality of micro-perforations do not penetrate an innermost lamina of the polymeric sheet.

29. The method of claim 16, wherein the insect is selected from the group consisting of stink bugs, shield bugs, squash bugs, and plant bugs, or any combination thereof.

30. The method of claim 16, wherein the insect is selected from a green stink bug (Acrosternum hilare (Say)); any of several species of Euschistus spp. including a brown stink bug (Euschistus servus (Say)), E. tristigmus, E. conspersus, E. variolarius, E. politus, or E. heros; a southern green stink bug (Nezara viridula (L.)); an eastern green stink bug (Nezara antennata); a spined soldier bug (Podisus maculiventris); an East Asian stink bug, a yellow-brown stink bug, a brown marmorated stink bug (Halyomorpha halys); a red-shouldered stink bug (Thyanta pallidovirens); a globular stink bug (Megacopta punctatissimum); a white-spotted stink bug (Eysarcoris ventralis); a fruit-piercing stink bug (Glaucias subpunctatus); a red-striped stink bug (Graphosoma rubrolineatum); a brown marmorated stink bug (Halyomorpha mista); a rice stink bug (Lagynotomus elongates, Oebalus pugnax); a two-spotted stink bug (Perillus bioculatus); a conchuela stink bug (Chlorochroa ligata); a Uhler's stink bug (Chlorochroa uhlerii); a Say's stink bug (Chlorochroa sayi); a brown-winged green stink bug (Plautia stali (Scott)); a boxelder bug (Boisea trivittata (Say)); a species of Banasa dimidiata (Say); a species of B. calva (Say); a species of B. euchlora Stål; a Harlequin bug (Murgantia histrionica); a Kudzu bug [Megacopta cribraria (Fabricius)]; a species of Anasa spp., including A. tristis (DeGeer); a species from the genus Leptoglossus; a species of Lygus spp., including a tarnished plant bug (L. lineolaris), a western tarnished plant bug (L. hesperus), a European tarnished plant bug (L. ruguilipnennis), a green leaf bug (L. lucorum); a species of apple dimpling bug (Campylomma spp.); a species of mosquito bug (Helopeltis spp.); a species of a honeylocust plant bug (Diaphnocoris spp.); a species of green mirid (Creontiades spp.); and a species of potato mirid (Calocoris spp).

31. A method for repelling insects, comprising:
releasing into a space a repellent composition comprising the Spined soldier bug aggregation pheromone; wherein the repellent composition comprises trans-2-hexenal, benzyl alcohol and α-terpineol in a controlled release device having at least one aperture configured to achieve a desired rate of release of the repellent composition in a volatilized state into the space; and
repelling from the space an insect belonging to the insect Pentatomoidea superfamily, including stink bugs, and shield bugs; the Superfamily Coreoidea, including squash bugs, or the family Miridae, including plant bugs, wherein each synthetic compound acts to repel the insect.

32. The method of claim 31, wherein the controlled release device comprises a polymeric sheet having a means for permitting the repellent composition in a volatilized state to pass therethrough.

33. The method of claim 32, wherein the polymeric sheet comprises a plurality of lamina.

34. The method of claim 33, wherein an innermost lamina of the plurality of lamina is semi-permeable such that the repellent composition in a volatilized state can pass through the innermost lamina.

35. The method of claim 32, wherein the means for permitting the repellent composition to pass therethrough comprises a plurality of micro-perforations.

36. The method of claim 35, wherein the polymeric sheet further comprises an innermost lamina and wherein at least some of the plurality of micro-perforations do not penetrate an innermost lamina of the polymeric sheet.

37. The method of claim 31, wherein the insect is selected from the group consisting of stink bugs, shield bugs, squash bugs, and plant bugs, or any combination thereof.

38. The method of claim 31, wherein the insect is selected from a green stink bug (Acrosternum hilare (Say)); any of several species of Euschistus spp. including a brown stink bug (Euschistus servus (Say)), E. tristigmus, E. conspersus, E. variolarius, E. politus, or E. heros; a southern green stink bug (Nezara viridula (L.)); an eastern green stink bug (Nezara antennata); a spined soldier bug (Podisus maculiventris); an East Asian stink bug, a yellow-brown stink bug, a brown marmorated stink bug (Halyomorpha halys); a red-shouldered stink bug (Thyanta pallidovirens); a globular stink bug (Megacopta punctatissimum); a white-spotted stink bug (Eysarcoris ventralis); a fruit-piercing stink bug (Glaucias subpunctatus); a red-striped stink bug (Graphosoma rubrolineatum); a brown marmorated stink bug (Halyomorpha mista); a rice stink bug (Lagynotomus elongates, Oebalus pugnax); a two-spotted stink bug (Perillus bioculatus); a conchuela stink bug (Chlorochroa ligata); a Uhler's stink bug (Chlorochroa uhlerii); a Say's stink bug (Chlorochroa sayi); a brown-winged green stink bug (Plautia stali (Scott)); a boxelder bug (Boisea trivittata (Say)); a species of Banasa dimidiata (Say); a species of B. calva (Say); a species of B. euchlora Stål; a Harlequin bug (Murgantia histrionica); a Kudzu bug [Megacopta cribraria (Fabricius)]; a species of Anasa spp., including A. tristis (DeGeer); a species from the genus Leptoglossus; a species of Lygus spp., including a tarnished plant bug (L. lineolaris), a western tarnished plant bug (L. hesperus), a European tarnished plant bug (L. ruguilipnennis), a green leaf bug (L. lucorum); a species of apple dimpling bug (Campylomma spp.); a species of mosquito bug (Helopeltis spp.); a species of a honeylocust plant bug (Diaphnocoris spp.); a species of green mirid (Creontiades spp.); and a species of potato mirid (Calocoris spp).

39. A method of controlling insects, comprising:
placing one or more insect repellent compositions, wherein the placement of the one or more repellent compositions is configured to push an insect in a direction of a location;
attracting the insect being pushed by the one or more repellent compositions to the location with an insect attractant composition; and
capturing the insect at the location, wherein the insect belongs to the order Heteroptera, the Pentatomoidea superfamily, including stink bugs, and shield bugs; the Superfamily Coreoidea, including squash bugs, or the insect family Miridae, including plant bugs.
